(19) United States
(12) Patent Application Publication
(10) Pub. No.: US 2024/0384003 A1
(43) Pub. Date: Nov. 21, 2024

(54) METHODS AND COMPOSITIONS FOR TREATING HEMATOLOGICAL MALIGNANCIES

(71) Applicant: PRECISION BIOLOGICS, INC., BETHESDA, MD (US)

(72) Inventors: Kwong Y. TSANG, Bethesda, MD (US); Massimo FANTINI, Bethesda, MD (US); Philip M. ARLEN, Bethesda, MD (US)

(21) Appl. No.: 18/290,553

(22) PCT Filed: May 19, 2022
(86) PCT No.: PCT/US2022/030028

§ 371 Date: Nov. 14, 2023

Related U.S. Application Data
(60) Provisional application No. 63/190,466, filed on May 19, 2021.

(51) Int. Cl. C07K 16/30; A61P 35/02; C07K 16/28; G01N 33/574

(52) U.S. Cl. CPC C07K 16/3007; A61P 35/02; C07K 16/2803; G01N 33/57426; C07K 2317/24; C07K 2317/31; C07K 2317/732

(57) ABSTRACT

NEO-201 has been shown to specifically bind and kill hematological malignancy cells, e.g., acute myeloid leukemia (AML), and multiple myeloma. Diagnostic methods, therapeutic methods, and combination therapies using NEO-201 optionally in combination with another agent are described. In some embodiments, the hematological malignancy is characterized by cancer cells which express O-glycans.

### CROSS-REFERENCE TO RELATED APPLICATION

This PCT application claims priority to U.S. Prov. Appl. No. 63/190,466, filed on May 19, 2021, the contents of which are incorporated by reference in their entirety.

### SEQUENCE LISTING INFORMATION

This application includes as part of its disclosure a biological sequence listing in the file named “4328204800.txt”, created on May 17, 2021, having a size of 32,432 bytes, which is hereby incorporated by reference in its entirety.

### BACKGROUND

The human carcinoembryonic antigen (CEA) family is a composed of 29 genes tandemly arranged on chromosome 19q13.2. Based on nucleotide homologies, these genes are classified into two major subfamilies, the CEACAM and pregnancy-specific glycoprotein subgroups. The CEACAM-encoded proteins include CEA (CEACAM5), CEA-related cell adhesion molecules (CEACAM1, CEACAM3, CEACAM4, CEACAM6, CEACAM7 and CEACAM8. CEACAM family belongs to the lg superfamily. Structurally, each of the human CEACAMs contain one N-terminal domain that includes 108-110 amino acid and is homologous to lg variable domains, followed by a different number (zero to six) of lg C2-type constant-like domains. The CEACAM proteins can interact homophilically and heterophilically with each other. CEACAM1 is a unique protein within this family because it contains an ITIM (immunoreceptor tyrosine-based inhibitory motif) like PD-1 in its cytoplasmic domain. This inhibitory effect is triggered by phosphorylation of tyrosine residues with the ITIM, which results in recruitment of the Src homology 2 domain-containing tyrosine phosphatase-1 and -2. The CEACAM1 protein is expressed on a variety of immune cells including monocytes, granulocytes, activated T cells, B cells and NK cells. CEACAM1 occurs as several isoforms, the two major ones being CEACAM1-L and CEACAM1-S that have long (L), or short(S) cytoplasmic domains, respectively. CEACAM1-S expression is totally lacking in human leukocytes. CEACAM1-L is expressed on subpopulation of activated human NK cells that are negative for CD16 but positive for CD56. Heterophilic interactions between CEA on tumor cells and CEACAM1 on NK cells inhibit NK cell cytotoxicity against tumor cells.

NEO-201 is a humanized IgG1 mAb that was generated against the Hollinshead allogeneic colorectal cancer vaccine platform (Hollinshead et al., Lancet. 1970; 1 (7658): 1191-1195; Hollinshead et al., Science. 1972; 177 (4052): 887-889). The immunogenic components of this vaccine were tumor-associated antigens (TAAs) that were derived from tumor membrane fractions pooled from surgically resected specimens from 79 patients with colon cancer (Hollinshead et al., Cancer. 1985; 56 (3): 480-489). These membrane fractions were semi-purified, screened for delayed-type hypersensitivity (DTH) in colon cancer patients versus healthy volunteers, and evaluated in clinical trials in patients with refractory colorectal cancer (Hollinshead et al., Cancer. 1985; 56 (3): 480-489; Hollinshead, U.S. Pat. No. 4,810,781, 1989; Bristol & Kantor, U.S. Pat. No. 7,829,678, 2010). These trials reported clinical benefit as defined by both antitumor response and significant prolongation in overall survival in patients that developed a sustained IgG response in addition to a cell-mediated response against the vaccine, thereby suggesting that the vaccine contained immunogenic components capable of generating antitumor antibodies (Hollinshead, Semin Surg Oncol. 1991 July-August; 7 (4): 199-210).

Prior work by the present inventors and others has indicated that NEO-201 binds a tumor-associated variants of CEACAM family members, particularly cancer-associated variants of CEACAM5 and CEACAM6 (Zeligs et al., Cancer Res. Jul. 1 2017 (77) (13 Supplement) 3025). NEO-201 is an lgG1 mAb that has been demonstrated to be reactive against certain carcinomas, but not reactive against most normal tissues. Functional analysis revealed that NEO-201 is capable of engaging innate immune effector mechanisms such as ADCC and CDC to kill tumor cells in vitro (Fantini et al., Front Immunol. 2018, 8, 1899; Fantini et al., Cancer Biother Radiopharm. 2019, 34, 147-159; Zeligs et al., Front Oncol. 2020, 10, 805). Additionally, NEO-201 can block the interaction between CEACAM5 on tumor cells and CEACAM1 on NK cells to reverse CEACAM1-dependent inhibition of NK cytotoxicity (Fantini et al., Cancer Biother Radiopharm. 2020, 35, 190-198). Previous studies showed that NEO-201 has also the ability to attenuate growth of human pancreatic tumor xenografts in mice and to prolong survival of ovarian cancer-bearing mice (Fantini et al., Front Immunol. 2018, 8, 1899). NEO-201 demonstrated safety/tolerability in non-human primates with a transient decrease in neutrophils being the only adverse effect observed (Zeligs et al., Front Oncol. 2020, 10, 805). Furthermore, we have demonstrated that NEO-201 binds specifically to tumor-associated CEACAM-5 and CEACAM-6 variants but not to those expressed on healthy tissues (Zeligs et al., Front Oncol. 2020, 10, 805). In this regard, we have also demonstrated that NEO-201 can block the interaction between CEACAM-5 expressed on tumor cells and CEACAM-1 expressed on NK cells to reverse CEACAM-1-dependent inhibition of NK cytotoxicity (Fantini et al., Cancer Biother Radiopharm. 2020, 35, 190-198). In addition, we have demonstrated that NEO-201 can bind to highly differentiated and most suppressive Treg cells in both healthy donors and cancer patients, and can eliminate them through CDC in vitro. An open label, first-in-human, phase 1, dose escalation study to determine the safety and maximal tolerated dose of the monoclonal antibody NEO-201 in adults with solid tumors that progressed following standard treatments has been completed at the NIH Clinical Center.

Applicant's prior U.S. Pat. Nos. 5,688,657, 7,314,622, 7,491,801, 7,763,720, 7,829,678, 8,470,326, 8,524,456, 8,535,667, 8,802,090, 9,034,588, 9,068,014, 9,371,375, 9,592,290, 9,718,866, and RE39,760, each of which is hereby incorporated by reference in its entirety, disclose various anti-cancer antibodies, cancer antigens, and related technologies. Particularly, certain of Applicant's prior patents disclose the use of NEO-201 in the diagnosis and treatment of colon and pancreas cancers. However, to Applicant's knowledge, the use of NEO-201 in the treatment of hematological malignancies has not been described previously.

### BRIEF DESCRIPTION

Applicants herein show for the first time that NEO-201 binds to certain hematological cells and can kill cells present in hematological malignancies. These results were unexpected, as the NEO-201 antigens (cancer-associated glycosylated variants of CEACAM5 and CEACAM6) were not previously known to be expressed by these cells, much less expressed at amounts high enough to permit killing of hematological cells by NEO-201. From the results herein it is concluded that NEO-201 can react against cells present in hematological malignancies and that the antigen bound by NEO-201 can be used as both a diagnostic marker and a therapeutic target in methods of detecting and/or treating hematological malignancies in subjects in need thereof.

### BRIEF DESCRIPTION OF THE FIGURES

FIG. 1A-1D: Staining of HL-60 cells by the NEO-201 antibody. Percentage of NEO-201 positive cells were determined by flow cytometry (right panels) compared to control unstained HL-60 cells (left panels).

FIG. 2: NEO-201 mediates ADCC against HL-60 (acute promyelocytic leukemia cell line or acute myeloid leukemia cell line) using PBMCs as effector cells. Results are presented as mean of % specific lysis±SD (Standard deviation) from 3 replicate wells in each experiment. *** statistically significant (p<0.001) by 2way ANOVA (NEO-201+PBMCs vs lgG1+PBMCs). Effector: Target cell ratios were 50:1 or 25:1 as labeled.

FIG. 3: NEO-201 mediates ADCC against HL-60 (acute promyelocytic leukemia cell line) using NK cells as effector cells. Results are presented as mean of % specific lysis±SD (Standard deviation) from 3 replicate wells in each experiment. *** statistically significant (p<0.001) by 2way ANOVA (NEO-201+NK cells vs lgG1+PBMCs). ** statistically significant (p<0.01) by 2way ANOVA (NEO-201+NK cells vs lgG1+PBMCs). Effector: Target cell ratios were 20:1 or 10:1 as labeled.

FIG. 4: Analysis of the expression of cell-surface antigen recognized by NEO-201 in hematological neoplastic cell lines. In the figure the data are presented as percentage of cells expressing the antigen recognized by NEO-201. Positivity was determined by using fluorescence minus one controls.

FIG. 5: Structures of o-glycan Cores found in mucins. In the table in the figure Gal is galactose; GalNAc is N-acetygalactosamine; GlcNAc is N-acetylglucosamine; Sial is sialic acid; Ser is serine; and Thr is threonine.

FIG. 6: O-glycan profile expressed by CFPAC-1 cell line.

FIGS. 7A to 7C: O-glycan profile expressed by human neutrophils.

FIG. 8: O-glycan profile expressed by U937, HL60 and K562 cell lines.

FIG. 9: Structures of O-GalNAc glycan Cores.

FIG. 10: An ELISA comparing NEO-201 binding to mammalian vs. bacterial rhCEACAM6.

FIG. 11: An array of O-glycan Array structures.

FIG. 12: Amino acid sequence of CEACAM6.

FIG. 13: Structure of different truncated C-terminus CEACAM6 gene constructs.

FIG. 14: Amino acid sequence of CEACAM5.

FIG. 15: Structure of different truncated C-terminus CEACAM5 gene constructs.

FIGS. 16-18: Binding studies using the anti-human IgG Fc Cy3 antibody detection method comparing the binding affinity of NEO-201 to an array of different O-glycans. As shown the background signal is low across the array and binding patterns with the anti-human lgG Fc Cy3 antibody detection method indicate that NEO-201 specifically binds to O-glycans 01, 02, 06, 23, 26 and 39 O-glycans, i.e., the detected binding of the anti-human lgG1 antibody to these O-glycans are real binding events, and do not represent non-specific binding. FIG. 16 shows the binding affinity of NEO-201 (100 mg/ml)+anti-hu lgG FcCy2 (20 mg/ml). FIG. 17 shows the binding affinity of NEO-201 (20 mg/ml)+anti-hu lgG FcCy2 (20 mg/ml). FIG. 18 shows the binding affinity of NEO-201 (4 mg/ml)+anti-hu lgG FcCy2 (20 mg/ml).

FIG. 19: Structures of 01, 02, 06, 023, 026 and 039 O-glycans.

FIG. 20: MS spectrum of O-glycans with annotations for CFPAC-1.

FIG. 21: MS spectrum of O-glycans with annotations for human neutrophils.

FIG. 22: MS spectrum of O-glycans with annotations for HL60 cell line.

FIG. 23: MS spectrum of O-glycans with annotations for U937 cell line.

FIG. 24: MS spectrum of O-glycans with annotations for K562 cell line.

FIG. 25: NEO-201 binding affinity to C-terminus truncated CEACAM6 proteins produced using a mammalian (CHO) expression system. In the experiments the expressed CEACAM6 variants were normalized by anti-human kappa chain ELISA, and two-fold serial diluted CEACAM6 variants coated wells were detected using NEO-201. As shown the OD values were decreased in the same concentration starting at construct of 318stop and reached lowest binding in the construct of CEACAM6 310stop. As further shown construct 320stop retains the full binding affinity to NEO-201 with the highest OD value in the same concentration of all expressed CEACAM6 variants.

FIG. 26: NEO-201 binding affinity to C-terminus truncated CEACAM5 proteins in a mammalian (CHO) expression system. The figure compares the binding curves of different truncated CEACAM5 proteins in ELISA binding assays.

FIG. 27: Flow cytometry experiments showing NEO-201 binding to different human carcinoma cell lines.

### DETAILED DESCRIPTION

In one aspect, the disclosure provides a method of treating or preventing a hematological malignancy, decreasing the burden of a hematological malignancy, or slowing the growth or proliferation rate of a hematological malignancy, comprising administering an effective amount of a NEO-201 antibody or an antibody that binds to the same epitope as NEO-201 or which competes with NEO-201 for binding to the same epitope as NEO-201 to a patient in need thereof.

In another aspect, the disclosure provides a method of potentiating an immune response against a hematological malignancy in a patient in need thereof, comprising administering an effective amount of a NEO-201 antibody or an antibody that binds to the same epitope as NEO-201 or which competes with NEO-201 for binding to the same epitope as NEO-201 to said patient.

In another aspect, the disclosure provides a method of stimulating regression of a hematological malignancy in a patient, comprising administering an effective amount of a NEO-201 antibody or an antibody that binds to the same epitope as NEO-201 or which competes with NEO-201 for binding to the same epitope as NEO-201 to said patient, thereby activating, enhancing, or stimulating anti-cancer immunity in said patient.

Said hematological malignancy may comprise a leukemia, lymphoma, multiple myeloma, or myelodysplastic syndrome. For example, said hematological malignancy may be selected from acute lymphocytic leukemia (ALL), acute myeloid leukemia (AML), Burkitt lymphoma (BL), chronic lymphocytic leukemia (CLL), chronic myeloid leukemia (CML), hairy cell leukemia (HCL), Hodgkin's lymphoma (HL), marginal zone lymphoma (MZL), multiple myeloma (MM), myelodysplastic syndromes (MDS), myeloma, non-Hodgkin's lymphoma (NHL), or T-cell lymphoma (TCL). In a preferred embodiment, said hematological malignancy may comprise a leukemia, more preferably acute myeloid leukemia (AML). In a particularly preferred embodiment, said hematological malignancy is acute promyelocytic leukemia.

In another aspect the disclosure provides a method of treating or preventing a hematological malignancy in a patient in need thereof, comprising administering to the patient an effective amount of an antibody or antibody fragment which binds to glycosylated CEACAM 5 and CEACAM6 but not to aglycosylated CEACAM 5 or aglycosylated CEACAM6, preferably wherein said antibody or antibody fragment recognizes an O-glycosylated epitope binding to the Threonine in the region of amino acids 310 to 318 (RTTVTTITV) of CEACAM5 and to the Threonine and Serine in the region of amino acids 312 to 320 (TVTMITVSG) of CEACAM6.

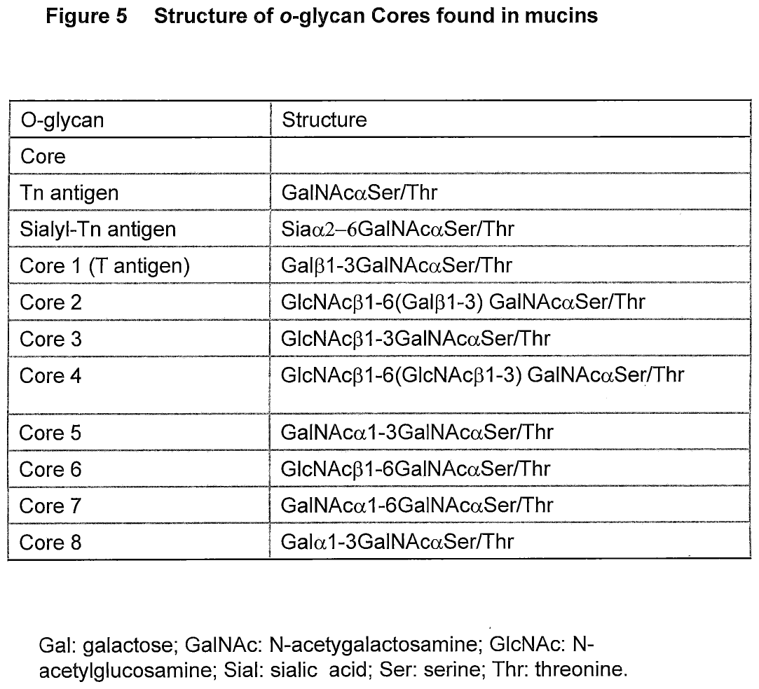

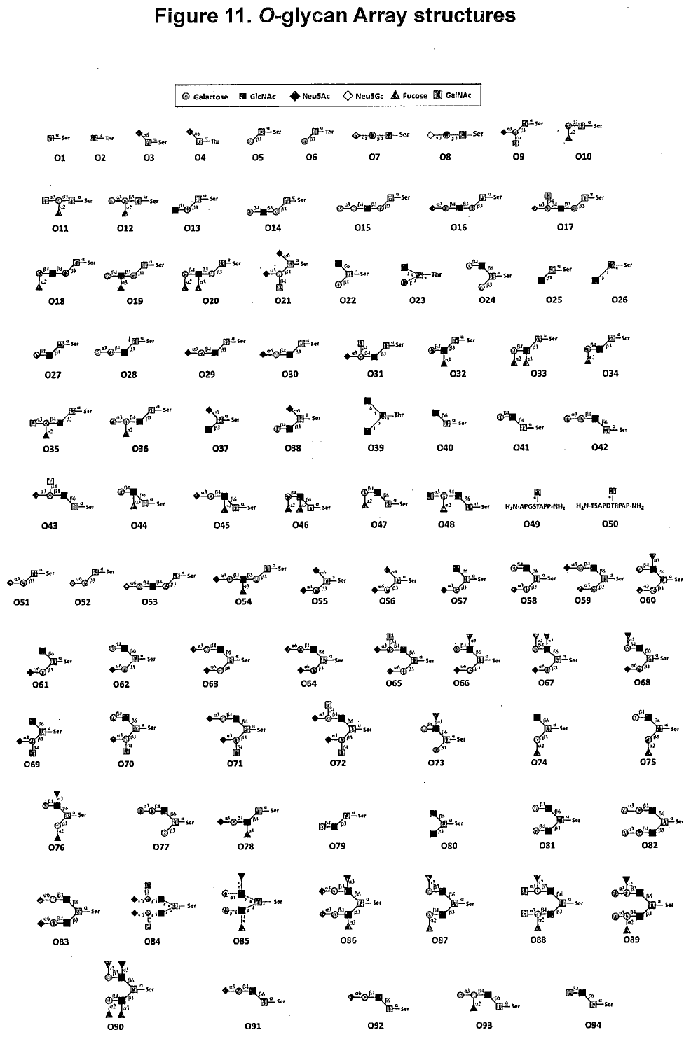

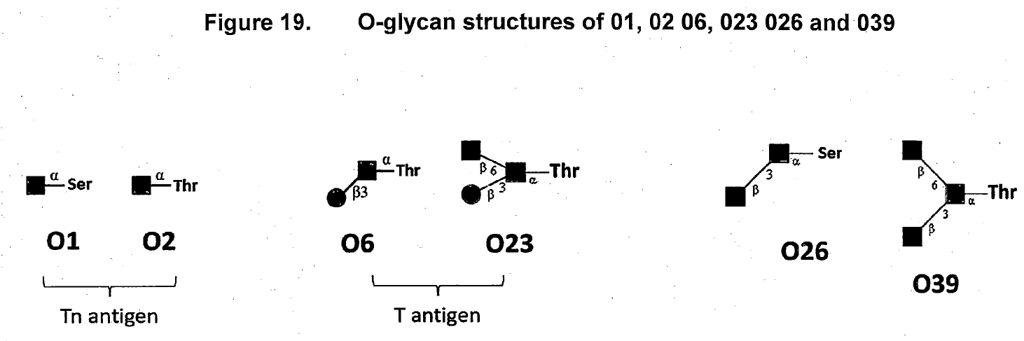

In another aspect the disclosure provides a method of treating or preventing a hematological malignancy in a patient in need thereof, comprising administering to the patient an effective amount of NEO-201 or an antigen binding fragment thereof, optionally wherein the hematological malignancy is characterized by cancer cells which express O-glycans selected from one or more of 01, 02, 06, 023, 026 and 039 O-glycans having the structure shown in the array in FIG. 11 and in FIG. 19 or the hematological malignancy is characterized by cancer cells which express 06, 01 or 02 O-glycans having the structure shown in the array in FIG. 11 and structures in FIG. 19, or the hematological malignancy is characterized by cancer cells which express 06 O-glycans as shown in the array in FIG. 11 and structures in FIG. 19, or the hematological malignancy is characterized by cancer cells which express Tn antigens or Core 1, 2, 4 or 4 O-glycans having the structures shown in FIG. 5.

In any of the previous methods, said hematological malignancy may comprise a leukemia, lymphoma, multiple myeloma, or myelodysplastic syndrome.

In any of the previous methods, said hematological malignancy further optionally may be selected from acute lymphocytic leukemia (ALL), acute myeloid leukemia (AML), Burkitt lymphoma (BL), chronic lymphocytic leukemia (CLL), chronic myeloid leukemia (CML), hairy cell leukemia (HCL), Hodgkin's lymphoma (HL), marginal zone lymphoma (MZL), multiple myeloma (MM), myelodysplastic syndromes (MDS), myeloma, non-Hodgkin's lymphoma (NHL), or T-cell lymphoma (TCL).

In any of the previous methods, said hematological malignancy optionally is a leukemia.

In any of the previous methods, said hematological malignancy optionally comprises acute myeloid leukemia (AML), and further optionally acute promyelocytic leukemia (APL).

In any of the previous methods, said hematological malignancy optionally is multiple myeloma.

In any of the previous methods, said hematological malignancy optionally expresses CEACAM5 and/or CEACAM6.

In any of the previous methods, said method optionally further comprises, prior to, or at the time of said administering said antibody one or more of the following:
(i) determining that said hematological malignancy is CEACAM5 and/or CEACAM6 positive;
(ii) determining that said hematological malignancy expresses (i) O-glycans selected from one or more of 01, 02, 06, 023, 026 and 039 having the structure shown in the array in FIG. 11 and in FIG. 19,
(iii) 06, 01 or 02 O-glycans having the structure shown in the array in FIG. 11 and in FIG. 19,
(iv) 06 O-glycans having the structure shown in the array in FIG. 11 and in FIG. 19 and/or
(v) In antigens or Core 1, 2, 3 or 4 O-glycans having the structures shown in FIG. 5.

In any of the previous methods, said NEO-201 antibody comprises one or more of the following:
(i) comprises the VH and VL CDR sequences contained in SEQ ID NO: 28 and SEQ ID NO: 29.
(ii) comprises a variable heavy chain sequence having at least 90% identity to SEQ ID NO: 38;
(iii) comprises a variable light chain sequence having at least 90% identity to SEQ ID NO: 39;
(iv) comprises a variable heavy chain sequence having at least 90% identity to SEQ ID NO: 38 and a variable light chain sequence having at least 90% identity to SEQ ID NO: 39;
(v) comprises a variable heavy chain sequence having at least 95% identity to SEQ ID NO: 38 and a variable light chain sequence having at least 95% identity to SEQ ID NO: 39;
(vi) comprises a variable heavy chain sequence identical to SEQ ID NO: 38 and a variable light chain sequence identical to SEQ ID NO: 39;
(vii) comprises a heavy chain sequence having at least 90% identity to amino acids 20-470 of SEQ ID NO: 28 and a light chain sequence having at least 90% identity to amino acids 20-233 of SEQ ID NO: 29;
(viii) comprises a heavy chain sequence having at least 95% identity to amino acids 20-470 of SEQ ID NO: 28 and a light chain sequence having at least 95% identity to amino acids 20-233 of SEQ ID NO: 29;
(ix) comprises a heavy chain sequence identical to amino acids 20-470 of SEQ ID NO: 28 and a light chain sequence identical to amino acids 20-233 of SEQ ID NO: 29;
(x) consists of the heavy chain sequence of amino acids 20-470 of SEQ ID NO: 28 and the light chain sequence of amino acids 20-233 of SEQ ID NO: 29;
(xi) comprises a human lgG1 constant domain;
(xii) is humanized;
(xiii) is conjugated to another moiety;
(xiv) is conjugated to another cytotoxic moiety, label, radioactive moiety, or affinity tag; or
(xv) is comprised in a chimeric antigen receptor (CAR);
(xvi) said antibody is a multispecific or bispecific antibody which targets at least one other antigen, optionally another tumor antigen or an antigen expressed on an immune cell, optionally wherein said other antigen is a checkpoint inhibitor or cytokine or hormone or growth factor.

In any of the previous methods, said antibody is optionally administered as an immune cell, optionally a T or NK cell, which immune cell expresses said antibody, fusion protein or a CAR comprising said antibody.

In another aspect the invention provides a method of killing hematological malignancy cells in vivo, comprising administering an effective amount of a NEO-201 antibody to a patient.

In another aspect the invention provides a method of killing hematological malignancy cells in vivo, comprising administering an effective amount of a NEO-201 antibody to a patient and at least one other active agent, wherein NEO-201 and the and at least one other active agent elicit an additive or synergistic effect in killing hematological malignancy cells.

In another aspect the invention provides a method of treating or preventing a hematological malignancy, decreasing the burden of a hematological malignancy, or slowing the growth or proliferation rate of a hematological malignancy, comprising administering an effective amount of a NEO-201 antibody or an antibody that binds to the same epitope as NEO-201 or which competes with NEO-201 for binding to the same epitope as NEO-201 to a patient in need thereof, wherein optionally in any of the previous methods said hematological malignancy is selected from leukemia, lymphoma, multiple myeloma, or myelodysplastic syndromes and further optionally wherein said hematological malignancy is selected from acute lymphocytic leukemia (ALL), acute myeloid leukemia (AML), Burkitt lymphoma (BL), chronic lymphocytic leukemia (CLL), chronic myeloid leukemia (CML), hairy cell leukemia (HCL), Hodgkin's lymphoma (HL), marginal zone lymphoma (MZL), multiple myeloma (MM), myelodysplastic syndromes (MDS), myeloma, non-Hodgkin's lymphoma (NHL), or T-cell lymphoma (TCL), still further optionally is a leukemia, optionally acute myeloid leukemia (AML), further optionally acute promyelocytic leukemia or is myeloma.

In any of the previous methods, said hematological malignancy optionally:
(i) expresses CEACAM5 or CEACAM6;
(ii) expresses O-glycans selected from one or more of 01, 02, 06, 023, 026 and 039 O-glycans having the structure shown in the array in FIG. 11 and in FIG. 19;
(iii) expresses 06, 01 or 02 O-glycans having the structure shown in the array in FIG. 11 and structures in FIG. 19; or
(iv) expresses 06 O-glycans as shown in the array in FIG. 11 and in FIG. 19 and/or
(v) expresses Tn antigens or Core 1, 2, 3 or 4 O-glycans having the structures shown in FIG. 5.

In any of the previous methods, optionally prior to, or at the time of said administering, determines whether said hematological malignancy:
(i) expresses CEACAM5 or CEACAM6;
(ii) expresses O-glycans selected from one or more of 01, 02, 06, 023, 026 and 039 O-glycans having the structure shown in the array in FIG. 11 and in FIG. 19;
(iii) expresses 06, 01 or 02 O-glycans having the structure shown in the array in FIG. 11 and structures in FIG. 19; or
(iv) expresses 06 O-glycans as shown in the array in FIG. 11 and in FIG. 19 and/or
(v) expresses Tn antigens or Core 1, 2, 3 or 4 O-glycans having the structures shown in FIG. 5.

In any of the previous methods, the NEO-201 antibody optionally:
(i) comprises the VH and VL CDR sequences contained in SEQ ID NO: 28 and SEQ ID NO: 29;
(ii) comprises a variable heavy chain sequence having at least 90% identity to SEQ ID NO: 38;
(iii) comprises a variable light chain sequence having at least 90% identity to SEQ ID NO: 39;
(iv) comprises a variable heavy chain sequence having at least 90% identity to SEQ ID NO: 38 and a variable light chain sequence having at least 90% identity to SEQ ID NO: 39;
(v) comprises a variable heavy chain sequence having at least 95% identity to SEQ ID NO: 38 and a variable light chain sequence having at least 95% identity to SEQ ID NO: 39;
(vi) comprises a variable heavy chain sequence identical to SEQ ID NO: 38 and a variable light chain sequence identical to SEQ ID NO: 39;
(vii) comprises a heavy chain sequence having at least 90% identity to amino acids 20-470 of SEQ ID NO: 28 and a light chain sequence having at least 90% identity to amino acids 20-233 of SEQ ID NO: 29;
(viii) comprises a heavy chain sequence having at least 95% identity to amino acids 20-470 of SEQ ID NO: 28 and a light chain sequence having at least 95% identity to amino acids 20-233 of SEQ ID NO: 29;
(ix) comprises a heavy chain sequence identical to amino acids 20-470 of SEQ ID NO: 28 and a light chain sequence identical to amino acids 20-233 of SEQ ID NO: 29;
(x) consists of the heavy chain sequence of amino acids 20-470 of SEQ ID NO: 28 and the light chain sequence of amino acids 20-233 of SEQ ID NO: 29;
(xi) comprises a human lgG1 constant domain;
(xii) is humanized;
(xiii) is conjugated to another moiety;
(xiv) is conjugated to another cytotoxic moiety, label, radioactive moiety, or affinity tag;
(xv) is comprised in a chimeric antigen receptor (CAR); or
(xvi) said antibody is a multispecific or bispecific antibody which targets at least one other antigen, optionally another tumor antigen or an antigen expressed on an immune cell, optionally wherein said other antigen is a checkpoint inhibitor or cytokine or hormone or growth factor.

In any of the previous methods, the method further optionally comprises administering another therapeutic agent to said patient, further optionally wherein said other agent is selected from (a) microtubule inhibitors, topoisomerase inhibitors, platins, alkylating agents, and anti-metabolites; (b) MK-2206, ON 013105, RTA 402, BI 2536, Sorafenib, ISIS-STAT3Rx, a microtubule inhibitor, a topoisomerase inhibitor, a platin, an alkylating agent, an anti-metabolite, paclitaxel, gemcitabine, doxorubicin, vinblastine, etoposide, 5-fluorouracil, carboplatin, altretamine, aminoglutethimide, amsacrine, anastrozole, azacitidine, bleomycin, busulfan, carmustine, chlorambucil, 2-chlorodeoxyadenosine, cisplatin, colchicine, cyclophosphamide, cytarabine, cytoxan, dacarbazine, dactinomycin, daunorubicin, docetaxel, estramustine phosphate, floxuridine, fludarabine, gentuzumab, hexamethylmelamine, hydroxyurea, ifosfamide, imatinib, interferon, irinotecan, lomustine, mechlorethamine, melphalen, 6-mercaptopurine, methotrexate, mitomycin, mitotane, mitoxantrone, pentostatin, procarbazine, rituximab, streptozocin, tamoxifen, temozolomide, teniposide, 6-thioguanine, topotecan, trastuzumab, vincristine, vindesine, and/or vinorelbine; (c) 1-D-ribofuranosyl-1,2,4-triazole-3 carboxamide, 9->2-hydroxy-ethoxy methylguanine, adamantanamine, 5-iodo-2′-deoxyuridine, trifluorothymidine, interferon, adenine arabinoside, protease inhibitors, thymidine kinase inhibitors, sugar or glycoprotein synthesis inhibitors, structural protein synthesis inhibitors, attachment and adsorption inhibitors, and nucleoside analogues such as acyclovir, penciclovir, valacyclovir, and ganciclovir; (d) a PD-1 inhibitor or anti-PD-1 antibody such as KEYTRUDA® (pembrolizumab), OPDIVO® (nivolumab), or LIBTAYO (cemiplimab); (e) a PD-L1 inhibitor or anti-PD-L1 antibody such as TECENTRIQ (atezolizumab), IMFINZI (durvalumab), or BAVENCIO (avelumab); or (f) a CTLA-4 inhibitor or anti-CTLA-4 antibody such as YERVOY® ipilimumab.

In any of the previous methods, optionally an anti-cancer vaccine is also administered to said patient.

In any of the previous methods, optionally said hematological malignancy cells are killed by CDC and/or ADCC.

In any of the previous methods, optionally said NEO-201 antibody is coupled to a cytotoxic moiety.

In another aspect the invention provides a method of killing hematological malignancy cells in vitro, comprising contacting said hematological malignancy cells with a NEO-201 antibody, said method optionally further comprising one or more of the following:
(i) contacting said hematological malignancy cells with complement; and/or
(ii) contacting said hematological malignancy cells with effector cells, optionally natural killer cells.

In another aspect the invention provides a method of detecting hematological malignancy cells, comprising detecting the expression of the NEO-201 antigen by said hematological malignancy cells, optionally wherein the level of hematological malignancy cells in a patient sample, such as a blood or biopsy sample, is used to diagnose cancer or determine cancer prognosis, wherein further optionally (i) said NEO-201 antibody is directly or indirectly coupled to a label, (ii) said detecting comprises cell sorting, further optionally fluorescence activated cell sorting.

In another aspect the invention provides a method of staining hematological malignancy cells in vivo or in an in vitro sample, comprising contacting said cells with a NEO-201 antibody, wherein optionally said NEO-201 antibody is directly or indirectly coupled to a label, further optionally wherein said sample is a tumor biopsy sample or comprises blood or bone marrow.

In another aspect the invention provides a method of isolating hematological malignancy cells, comprising isolating cells that express the NEO-201 target antigen, which optionally comprises contacting a sample containing hematological malignancy cells with a NEO-201 antibody, further optionally wherein said NEO-201 antibody is directly or indirectly labeled, wherein optionally said sample is or comprises blood or bone marrow, further optionally separating NEO-201 positive hematological malignancy cells from NEO-201 negative cells, further optionally by cell sorting, further optionally by fluorescence activated cell sorting.

In another aspect the invention provides a method of isolating hematological malignancy cells, by contacting sample with a support comprising a NEO-201 antibody, whereby said hematological malignancy cells are retained on said support.

In any of the previous methods said NEO-201 antibody preferably:
(i) comprises the VH and VL CDR sequences contained in SEQ ID NO: 28 and SEQ ID NO: 29;
(ii) comprises a variable heavy chain sequence having at least 90% identity to SEQ ID NO: 38;
(iii) comprises a variable light chain sequence having at least 90% identity to SEQ ID NO: 39;
(iv) comprises a variable heavy chain sequence having at least 90% identity to SEQ ID NO: 38 and a variable light chain sequence having at least 90% identity to SEQ ID NO: 39;
(v) comprises a variable heavy chain sequence having at least 95% identity to SEQ ID NO: 38 and a variable light chain sequence having at least 95% identity to SEQ ID NO: 39;
(vi) comprises a variable heavy chain sequence identical to SEQ ID NO: 38 and a variable light chain sequence identical to SEQ ID NO: 39;
(vii) comprises a heavy chain sequence having at least 90% identity to amino acids 20-470 of SEQ ID NO: 28 and a light chain sequence having at least 90% identity to amino acids 20-233 of SEQ ID NO: 29;
(viii) comprises a heavy chain sequence having at least 95% identity to amino acids 20-470 of SEQ ID NO: 28 and a light chain sequence having at least 95% identity to amino acids 20-233 of SEQ ID NO: 29;
(ix) comprises a heavy chain sequence identical to amino acids 20-470 of SEQ ID NO: 28 and a light chain sequence identical to amino acids 20-233 of SEQ ID NO: 29;
(x) consists of the heavy chain sequence of amino acids 20-470 of SEQ ID NO: 28 and the light chain sequence of amino acids 20-233 of SEQ ID NO: 29;
(xi) comprises a human lgG1 constant domain;
(xii) is humanized;
(xiii) is conjugated to another moiety;
(xiv) is conjugated to another cytotoxic moiety, label, radioactive moiety, or affinity tag;
(xv) is comprised in a chimeric antigen receptor (CAR); or
(xvi) said antibody is a multispecific or bispecific antibody which targets at least one other antigen, optionally another tumor antigen or an antigen expressed on an immune cell, optionally wherein said other antigen is a checkpoint inhibitor or cytokine or hormone or growth factor.

Additionally, in any of the foregoing or following methods, said NEO-201 antibody may compete with the antibody comprising the heavy chain of SEQ ID NO: 28 and the light chain of SEQ ID NO: 29 for binding to the NEO-201 antigen.

### Definitions

Unless defined otherwise, all technical and scientific terms used herein have the same meaning as those commonly understood by one of ordinary skill in the art to which this invention belongs. Although methods and materials similar or equivalent to those described herein may be used in the invention or testing of the present invention, suitable methods and materials are described herein. The materials, methods and examples are illustrative only, and are not intended to be limiting.

As used in the description herein and throughout the claims that follow, the meaning of “a,” “an,” and “the” includes plural reference unless the context clearly dictates otherwise.

“Acute myeloid leukemia” or “AML” herein includes all types of AML. For example, according to the French-American-British (FAB) classification of AML, AML herein specifically includes AML subtypes, M0 through M7, i.e., M0 (Undifferentiated acute myeloblastic leukemia), M1 (Acute myeloblastic leukemia with minimal maturation), M2 (Acute myeloblastic leukemia with maturation), M3 (Acute promyelocytic leukemia (APL)), M4 (Acute myelomonocytic leukemia), M4 eos (Acute myelomonocytic leukemia with eosinophilia), M5 (Acute monocytic leukemia), M6 (Acute erythroid leukemia) and M7 (Acute megakaryoblastic leukemia). According to the WHO system classification of AML, AML herein specifically includes AML with certain genetic abnormalities (gene or chromosome changes), e.g., AML with a translocation between chromosomes 8 and 21, AML with a translocation or inversion in chromosome 16, APL with the PML-RARA fusion gene, AML with a translocation between chromosomes 9 and 11, AML with a translocation between chromosomes 6 and 9, AML with a translocation or inversion in chromosome 3, AML (megakaryoblastic) with a translocation between chromosomes 1 and 22, AML with the BCR-ABL1 (BCR-ABL) fusion gene, AML with mutated NPM1 gene, AML with biallelic mutations of the CEBPA gene (i.e., mutations in both copies of the gene), AML with mutated RUNX1 gene, AML with myelodysplasia-related changes, AML related to previous chemotherapy or radiation, AML with minimal differentiation (FAB M0), AML without maturation (FAB M1), AML with maturation (FAB M2), Acute myelomonocytic leukemia (FAB M4), Acute monoblastic/monocytic leukemia (FAB M5), Pure erythroid leukemia (FAB M6), Acute megakaryoblastic leukemia (FAB M7), Acute basophilic leukemia, Acute panmyelosis with fibrosis, Myeloid sarcoma (also known as granulocytic sarcoma or chloroma), and Myeloid proliferations related to Down syndrome. Also AML herein includes AML-like conditions, i.e., undifferentiated and bi-phenotypic acute leukemias which are not strictly AML, but are leukemias that have both lymphocytic and myeloid features which are sometimes referred to as mixed phenotype acute leukemias (MPALs).

“Amino acid,” as used herein refers broadly to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The terms “NK-depleted” or “natural killer-depleted” as used herein refer to a patient having low natural killer (NK) cell levels relative to the normal range. NK cells are a cytotoxic innate immune lymphocyte. Typically, NK cells comprise 5-20% of the peripheral blood mononuclear cells (PBMCs) in a healthy individual. A patient having NK cells comprising less than 5% of the PMBCs is referred to as NK-depleted. Additionally, a patient is referred to as severely NK-cell depleted if NK cells comprising less than 3% of the PMBCs. Additionally, in normal individuals, up to 90% of PBMC NK cells are CD56^{dim}CD16+NK cells, and these are considered the most cytotoxic subset. If less than 70% of PBMC NK cells are CD56^{dim}CD16+NK cells, then the patient is referred to as NK-depleted. Additionally, if less than 50% of PBMC NK cells are CD56^{dim}CD16+NK cells, then the patient is referred to as severely NK-depleted. A given patient may be referred to as NK-depleted or severely NK-depleted based on meeting either or both of these individual criteria. Generally speaking, a patient's status as NK-depleted or severely NK-depleted is determined by testing a sample taken from the patient, e.g., a blood sample, e.g., a sample obtained and tested within one or two weeks prior. A patient's status as NK-depleted or severely NK-depleted may also be inferred from a disease diagnosis and/or a course of treatment that is associated with such depletion of NK cells.

“Antibody,” as used herein, refers broadly to any polypeptide chain-containing molecular structure with a specific shape that fits to and recognizes an epitope, where one or more non-covalent binding interactions stabilize the complex between the molecular structure and the epitope. The archetypal antibody molecule is the immunoglobulin, and all types of immunoglobulins, lgG, IgM, IgA, IgE, IgD, from all sources, e.g., human, rodent, rabbit, cow, sheep, pig, dog, chicken, are considered to be “antibodies.” Antibodies include but are not limited to chimeric antibodies, human antibodies and other non-human mammalian antibodies, humanized antibodies, single chain antibodies (scFvs), camelbodies, nanobodies, IgNAR (single-chain antibodies derived from sharks), small-modular immunopharmaceuticals (SMIPs), and antibody fragments (e.g., Fabs, Fab′, F(ab′) 2 and other antibody fragments). Numerous antibody coding sequences have been described; and others may be raised by methods well-known in the art. See Streltsov, et al. (2005) Protein Sci. 14 (11): 2901-9; Greenberg, et al. (1995) Nature 374 (6518): 168-173; Nuttall, et al. (2001) Mol Immunol. 38 (4): 313-26; Hamers-Casterman, et al. (1993) Nature 363 (6428): 446-8; Gill, et al. (2006) Curr Opin Biotechnol. 17 (6): 653-8.

“NEO-201 antibody” refers to an antibody containing the heavy and light chains of SEQ ID NOs: 28 and 29 or the variable regions optionally together with the constant regions contained therein, as well as fragments and variants thereof. Such variants include sequences containing one, two, three, four, five or preferably all six of the CDR sequences contained in SEQ ID NO: 28 and SEQ ID NO: 29, i.e., the heavy chain CDR1 of SEQ ID NO: 32, the heavy chain CDR2 of SEQ ID NO: 33, the heavy chain CDR3 of SEQ ID NO: 34, the light chain CDR1 of SEQ ID NO: 35, the light chain CDR2 of SEQ ID NO: 36, and the light chain CDR3 of SEQ ID NO: 37. Such variants also include antibodies that compete with NEO-201 for binding to the NEO-201 antigen. Said antibody may be humanized. Said antibody may be expressed containing one or more leader sequences, which may be removed during expression and/or processing and secretion of the antibody. Said antibody may be presented in a monovalent, bivalent, or higher multivalent format, including without limitation a bispecific or multispecific antibody containing said NEO-201 antibody sequence and a binding fragment of a different antibody. Typically said antibody specifically binds to carcinoma cells and competes for binding to carcinoma cells with an antibody comprising the variable heavy chain of SEQ ID NO: 38 and variable light chain of SEQ ID NO: 39, or comprising the heavy chain of SEQ ID NO: 28 and light chain of SEQ ID NO: 29. One or more of those CDR sequences contained in SEQ ID NO: 28 and/or SEQ ID NO: 29 may be substituted with a variant sequence, such as the light chain CDR1 of SEQ ID NO: 1 or 4; light chain CDR2 of SEQ ID NO: 2 or 5; light chain CDR3 of SEQ ID NO: 3 or 6; heavy chain CDR1 of SEQ ID NO: 7; heavy chain CDR2 of SEQ ID NO: 8, 10, 30, or 31; heavy chain CDR3 of SEQ ID NO: 9 or 11; or SEQ ID NOs: 30-31. The light chain may comprise the CDRs contained in the light chain sequence of SEQ ID NO: 14, 16, 17, 18, 19, 20, 21, or 29. The heavy chain may comprise the CDRs contained in the heavy chain sequence of SEQ ID NO: 15, 22, 23, 24, 25, 26, 27, or 29. Said antibody may comprise a variable heavy chain sequence having at least 75%, 80%, 85%, 90%, 95%, 96%, 97%, 98%, or 99% identity to SEQ ID NO: 38, and/or a variable light chain sequence having at least 75%, 80%, 85%, 90%, 95%, 96%, 97%, 98%, or 99% identity to SEQ ID NO: 39, optionally wherein said heavy and/or light chain sequence contains one, two, three, four, five or preferably all six of the CDR sequences contained in SEQ ID NO: 28 and SEQ ID NO: 29, i.e., the heavy chain CDR1 of SEQ ID NO: 32, the heavy chain CDR2 of SEQ ID NO: 33, the heavy chain CDR3 of SEQ ID NO: 34, the light chain CDR1 of SEQ ID NO: 35, the light chain CDR2 of SEQ ID NO: 36, and the light chain CDR3 of SEQ ID NO: 37. Said antibody may be conjugated to another moiety, such as a cytotoxic moiety, radioactive moiety, label, or purification tag.

“Antigen,” as used herein, refers broadly to a molecule or a portion of a molecule capable of being bound by an antibody which is additionally capable of inducing an animal to produce an antibody capable of binding to an epitope of that antigen. An antigen may have one epitope, or have more than one epitope. The specific reaction referred to herein indicates that the antigen will react, in a highly selective manner, with its corresponding antibody and not with the multitude of other antibodies which may be evoked by other antigens. Antigens may be tumor specific (e.g., expressed by neoplastic cells of pancreatic and colon carcinoma.)

“Cancer,” as used herein, refers broadly to any neoplastic disease (whether invasive or metastatic) characterized by abnormal and uncontrolled cell division causing malignant growth or tumor.

“Cancer vaccine,” as used herein, refers to an immunogenic composition that elicits or is intended to elicit an immune response against a cancer cell.

“Chimeric antibody,” as used herein, refers broadly to an antibody molecule in which the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug; or the variable region, or a portion thereof, is altered, replaced or exchanged with a variable region having a different or altered antigen specificity.

“Conservatively modified variants,” as used herein, applies to both amino acid and nucleic acid sequences, and with respect to particular nucleic acid sequences, refers broadly to conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) may be modified to yield a functionally identical molecule.

“Complementarity determining region,” “hypervariable region,” or “CDR,” as used herein, refers broadly to one or more of the hyper-variable or complementarily determining regions (CDRs) found in the variable regions of light or heavy chains of an antibody. See Kabat, et al. (1987) “Sequences of Proteins of Immunological Interest” National Institutes of Health, Bethesda, MD. These expressions include the hypervariable regions as defined by Kabat, et al. (1983) “Sequences of Proteins of Immunological Interest” U.S. Dept. of Health and Human Services or the hypervariable loops in 3-dimensional structures of antibodies. Chothia and Lesk (1987) J Mol. Biol. 196:901-917. The CDRs in each chain are held in close proximity by framework regions and, with the CDRs from the other chain, contribute to the formation of the antigen binding site. Within the CDRs there are select amino acids that have been described as the selectivity determining regions (SDRs) which represent the critical contact residues used by the CDR in the antibody-antigen interaction. Kashmiri (2005) Methods 36:25-34.

“Control amount,” as used herein, refers broadly to a marker can be any amount or a range of amounts to be compared against a test amount of a marker. For example, a control amount of a marker may be the amount of a marker in a patient with a particular disease or condition or a person without such a disease or condition. A control amount can be either in absolute amount (e.g., microgram/ml) or a relative amount (e.g., relative intensity of signals).

“Differentially present,” as used herein, refers broadly to differences in the quantity or quality of a marker present in a sample taken from patients having a disease or condition as compared to a comparable sample taken from patients who do not have one of the diseases or conditions. For example, a nucleic acid fragment may optionally be differentially present between the two samples if the amount of the nucleic acid fragment in one sample is significantly different from the amount of the nucleic acid fragment in the other sample, for example as measured by hybridization and/or NAT-based assays. A polypeptide is differentially present between the two samples if the amount of the polypeptide in one sample is significantly different from the amount of the polypeptide in the other sample. It should be noted that if the marker is detectable in one sample and not detectable in the other, then such a marker may be considered to be differentially present. Optionally, a relatively low amount of up-regulation may serve as the marker.

“Diagnostic,” as used herein, refers broadly to identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

“Diagnosing,” as used herein refers broadly to classifying a disease or a symptom, determining a severity of the disease, monitoring disease progression, forecasting an outcome of a disease and/or prospects of recovery. The term “detecting” may also optionally encompass any of the foregoing. Diagnosis of a disease according to the present invention may, in some embodiments, be affected by determining a level of a polynucleotide or a polypeptide of the present invention in a biological sample obtained from the subject, wherein the level determined can be correlated with predisposition to, or presence or absence of the disease. It should be noted that a “biological sample obtained from the subject” may also optionally comprise a sample that has not been physically removed from the subject.

“Effective amount,” as used herein, refers broadly to the amount of a compound, antibody, antigen, or cells that achieves a desired result. An “effective amount” when administered to a patient for treating a disease, is sufficient to effect such treatment for the disease. The effective amount may be an amount effective for prophylaxis, and/or an amount effective for prevention. The effective amount may be an amount effective to reduce, an amount effective to prevent the incidence of signs/symptoms, to reduce the severity of the incidence of signs/symptoms, to eliminate the incidence of signs/symptoms, to slow the development of the incidence of signs/symptoms, to prevent the development of the incidence of signs/symptoms, and/or effect prophylaxis of the incidence of signs/symptoms. The “effective amount” may vary depending on the disease and its severity and the age, weight, medical history, susceptibility, and pre-existing conditions, of the patient to be treated. The term “effective amount” is synonymous with “therapeutically effective amount” for purposes of this disclosure.

“Expression vector,” as used herein, refers broadly to any recombinant expression system for the purpose of expressing a nucleic acid sequence of the present disclosure in vitro or in vivo, constitutively or inducibly, in any cell, including prokaryotic, yeast, fungal, plant, insect or mammalian cell. The term includes linear or circular expression systems. The term includes expression systems that remain episomal or integrate into the host cell genome. The expression systems can have the ability to self-replicate or not, i.e., drive only transient expression in a cell. The term includes recombinant expression cassettes which contain only the minimum elements needed for transcription of the recombinant nucleic acid.

“Framework region” or “FR,” as used herein, refers broadly to one or more of the framework regions within the variable regions of the light and heavy chains of an antibody. See Kabat, et al. (1987) “Sequences of Proteins of Immunological Interest,” National Institutes of Health, Bethesda, MD. These expressions include those amino acid sequence regions interposed between the CDRs within the variable regions of the light and heavy chains of an antibody.

“Hematological malignancy” refers to forms of cancer that begin in blood-forming tissue, such as the bone marrow, or in the cells of the immune system. Examples of hematological malignancies include leukemia, lymphoma, multiple myeloma, and myelodysplastic syndromes (MDS). More specific examples of hematological malignancies include but are not limited to marginal zone lymphoma (MZL) (including splenic marginal zone lymphoma (SMZL)), Burkitt lymphoma (BL), multiple myeloma (MM) (including plasma cell leukemia (PCL) and myeloma extramedullary disease (EMD)), myelodysplastic syndromes (MDS), acute myeloid leukemia (AML) (including B-cell AML), acute lymphocytic leukemia (ALL), T-cell lymphoma (TCL) (including anaplastic large cell lymphoma (ALCL) and Sezary Syndrome), and Hodgkin's lymphoma (HL).

“Acute myeloid leukemia” or “AML” includes all types of AML. According to the French-American-British (FAB) classification of AML, AML herein specifically includes AML subtypes, M0 through M7, i.e., M0 (Undifferentiated acute myeloblastic leukemia), M1 (Acute myeloblastic leukemia with minimal maturation), M2 (Acute myeloblastic leukemia with maturation), M3 (Acute promyelocytic leukemia (APL)), M4 (Acute myelomonocytic leukemia), M4 eos (Acute myelomonocytic leukemia with eosinophilia), M5 (Acute monocytic leukemia), M6 (Acute erythroid leukemia) and M7 (Acute megakaryoblastic leukemia).

According to the WHO system classification of AML, AML herein specifically includes AML with certain genetic abnormalities (gene or chromosome changes), e.g., AML with a translocation between chromosomes 8 and 21, AML with a translocation or inversion in chromosome 16, APL with the PML-RARA fusion gene, AML with a translocation between chromosomes 9 and 11, AML with a translocation between chromosomes 6 and 9, AML with a translocation or inversion in chromosome 3, AML (megakaryoblastic) with a translocation between chromosomes 1 and 22, AML with the BCR-ABL1 (BCR-ABL) fusion gene, AML with mutated NPM1 gene, AML with biallelic mutations of the CEBPA gene (i.e., mutations in both copies of the gene), AML with mutated RUNX1 gene, AML with myelodysplasia-related changes, AML related to previous chemotherapy or radiation, AML with minimal differentiation (FAB M0), AML without maturation (FAB M1), AML with maturation (FAB M2), Acute myelomonocytic leukemia (FAB M4), Acute monoblastic/monocytic leukemia (FAB M5), Pure erythroid leukemia (FAB M6), Acute megakaryoblastic leukemia (FAB M7), Acute basophilic leukemia, Acute panmyelosis with fibrosis, Myeloid sarcoma (also known as granulocytic sarcoma or chloroma), and Myeloid proliferations related to Down syndrome. Also AML herein includes AML-like conditions, i.e., undifferentiated and bi-phenotypic acute leukemias which are not strictly AML, but are leukemias that have both lymphocytic and myeloid features which are sometimes referred to as mixed phenotype acute leukemias (MPALs).

“Heterologous,” as used herein, refers broadly to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

“High affinity,” as used herein, refers broadly to an antibody having a KD of at least 10⁻⁸ M, more preferably at least 10⁻⁹ M and even more preferably at least 10⁻¹⁰ M for a target antigen. However, “high affinity” binding can vary for other antibody isotypes. For example, “high affinity” binding for an IgM isotype refers to an antibody having a KD of at least 10⁻⁷ M, more preferably at least 10⁻⁸ M.

“Homology,” as used herein, refers broadly to a degree of similarity between a nucleic acid sequence and a reference nucleic acid sequence or between a polypeptide sequence and a reference polypeptide sequence. Homology may be partial or complete. Complete homology indicates that the nucleic acid or amino acid sequences are identical. A partially homologous nucleic acid or amino acid sequence is one that is not identical to the reference nucleic acid or amino acid sequence. The degree of homology can be determined by sequence comparison. The term “sequence identity” may be used interchangeably with “homology.”

“Host cell,” as used herein, refers broadly to a cell that contains an expression vector and supports the replication or expression of the expression vector. Host cells may be prokaryotic cells such as E. coli, or eukaryotic cells such as yeast, insect (e.g., SF9), amphibian, or mammalian cells such as CHO, HeLa, HEK-293, e.g., cultured cells, explants, and cells in vivo.

“Hybridization,” as used herein, refers broadly to the physical interaction of complementary (including partially complementary) polynucleotide strands by the formation of hydrogen bonds between complementary nucleotides when the strands are arranged antiparallel to each other.

“K-assoc” or “Ka”, as used herein, refers broadly to the association rate of a particular antibody-antigen interaction, whereas the term “Kdiss” or “Kd,” as used herein, refers to the dissociation rate of a particular antibody-antigen interaction. The term “KD”, as used herein, is intended to refer to the dissociation constant, which is obtained from the ratio of Kd to Ka (i.e., Kd/Ka) and is expressed as a molar concentration (M). KD values for antibodies can be determined using methods well established in the art.

“Immunoassay,” as used herein, refers broadly to an assay that uses an antibody to specifically bind an antigen. The immunoassay may be characterized by the use of specific binding properties of a particular antibody to isolate, target, and/or quantify the antigen.

“Isolated,” as used herein, refers broadly to material removed from its original environment in which it naturally occurs, and thus is altered by the hand of man from its natural environment. Isolated material may be, for example, exogenous nucleic acid included in a vector system, exogenous nucleic acid contained within a host cell, or any material which has been removed from its original environment and thus altered by the hand of man (e.g., “isolated antibody”).

“Label” or a “detectable moiety” as used herein, refers broadly to a composition detectable by spectroscopic, photochemical, biochemical, immunochemical, chemical, or other physical means.

“Low stringency,” “medium stringency,” “high stringency,” or “very high stringency conditions,” as used herein, refers broadly to conditions for nucleic acid hybridization and washing. Guidance for performing hybridization reactions can be found in Ausubel, et al. (2002) Short Protocols in Molecular Biology (5th Ed.) John Wiley & Sons, NY. Exemplary specific hybridization conditions include but are not limited to: (1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); (2) medium stringency hybridization conditions in 6×SSC at about 45° C., followed by one or more washes in 0.2×SSC, 0.1% SDS at 60° C.; (3) high stringency hybridization conditions in 6×SSC at about 45° C., followed by one or more washes in 0.2×SSC, 0.1% SDS at 65° C.; and (4) very high stringency hybridization conditions are 0.5M sodium phosphate, 7% SDS at 65° C., followed by one or more washes at 0.2×SSC, 1% SDS at 65° C.

The term “low level” or “low” as used in relation to a marker such as CD127 is well known in the art and refers to the expression level of the cell marker of interest (e.g., CD 127), in that the expression level of the cell marker is low by comparison with the expression level of that cell marker in other cells in a population of cells being analyzed as a whole. More particularly, the term “low” refers to a distinct population of cells that express the cell marker at a lower level than one or more other distinct population of cells. Accordingly CD127^{low} refers to cells of a type that stains slightly or dully when contacted with a labeled CD127 antibody, e.g., at a level that is higher than a CD127-subpopulation but lower than the CD127+ subpopulation.

“Mammal,” as used herein, refers broadly to any and all warm-blooded vertebrate animals of the class Mammalia, including humans, characterized by a covering of hair on the skin and, in the female, milk-producing mammary glands for nourishing the young. Examples of mammals include but are not limited to alpacas, armadillos, capybaras, cats, camels, chimpanzees, chinchillas, cattle, dogs, goats, gorillas, hamsters, horses, humans, lemurs, llamas, mice, non-human primates, pigs, rats, sheep, shrews, squirrels, and tapirs. Mammals include but are not limited to bovine, canine, equine, feline, murine, ovine, porcine, primate, and rodent species. Mammal also includes any and all those listed on the Mammal Species of the World maintained by the National Museum of Natural History, Smithsonian Institution in Washington DC.

“Nucleic acid” or “nucleic acid sequence,” as used herein, refers broadly to a deoxy-ribonucleotide or ribonucleotide oligonucleotide in either single- or double-stranded form. The term encompasses nucleic acids, i.e., oligonucleotides, containing known analogs of natural nucleotides. The term also encompasses nucleic-acid-like structures with synthetic backbones. Unless otherwise indicated, a particular nucleic acid sequence also implicitly encompasses conservatively modified variants thereof (e.g., degenerate codon substitutions) and complementary sequences, as well as the sequence explicitly indicated. The term nucleic acid is used interchangeably with gene, cDNA, mRNA, oligonucleotide, and polynucleotide.

“Operatively linked”, as used herein, refers broadly to when two DNA fragments are joined such that the amino acid sequences encoded by the two DNA fragments remain in-frame.

“Paratope,” as used herein, refers broadly to the part of an antibody which recognizes an antigen (e.g., the antigen-binding site of an antibody.) Paratopes may be a small region (e.g., 15-22 amino acids) of the antibody's Fv region and may contain parts of the antibody's heavy and light chains. See Goldsby, et al. Antigens (Chapter 3) Immunology (5th Ed.) New York: W. H. Freeman and Company, pages 57-75.

“Patient,” as used herein, refers broadly to any animal who is in need of treatment either to alleviate a disease state or to prevent the occurrence or reoccurrence of a disease state. Also, “Patient” as used herein, refers broadly to any animal who has risk factors, a history of disease, susceptibility, symptoms, signs, was previously diagnosed, is at risk for, or is a member of a patient population for a disease. The patient may be a clinical patient such as a human or a veterinary patient such as a companion, domesticated, livestock, exotic, or zoo animal. The term “subject” may be used interchangeably with the term “patient”. In preferred embodiments of the inventions disclosed herein, the patient is a human.

“Polypeptide,” “peptide” and “protein,” are used interchangeably and refer broadly to a polymer of amino acid residues. The terms apply to amino acid polymers in which one or more amino acid residue is an analog or mimetic of a corresponding naturally occurring amino acid, as well as to naturally occurring amino acid polymers. The terms apply to amino acid polymers in which one or more amino acid residue is an artificial chemical mimetic of a corresponding naturally occurring amino acid, as well as to naturally occurring amino acid polymers and non-naturally occurring amino acid polymer. Polypeptides can be modified, e.g., by the addition of carbohydrate residues to form glycoproteins. The terms “polypeptide,” “peptide” and “protein” include glycoproteins, as well as non-glycoproteins.

“Promoter,” as used herein, refers broadly to an array of nucleic acid sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation.

“Prophylactically effective amount,” as used herein, refers broadly to the amount of a compound that, when administered to a patient for prophylaxis of a disease or prevention of the reoccurrence of a disease, is sufficient to effect such prophylaxis for the disease or reoccurrence. The prophylactically effective amount may be an amount effective to prevent the incidence of signs and/or symptoms. The “prophylactically effective amount” may vary depending on the disease and its severity and the age, weight, medical history, predisposition to conditions, preexisting conditions, of the patient to be treated.

“Prophylaxis,” as used herein, refers broadly to a course of therapy where signs and/or symptoms are not present in the patient, are in remission, or were previously present in a patient. Prophylaxis includes preventing disease occurring subsequent to treatment of a disease in a patient. Further, prevention includes treating patients who may potentially develop the disease, especially patients who are susceptible to the disease (e.g., members of a patent population, those with risk factors, or at risk for developing the disease).

“Recombinant” as used herein, refers broadly with reference to a product, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

“Specifically (or selectively) binds” to an antibody or “specifically (or selectively) immunoreactive with,” or “specifically interacts or binds,” as used herein, refers broadly to a protein or peptide (or other epitope), refers, in some embodiments, to a binding reaction that is determinative of the presence of the protein in a heterogeneous population of proteins and other biologics. For example, under designated immunoassay conditions, the specified antibodies bind to a particular protein at least two times greater than the background (non-specific signal) and do not substantially bind in a significant amount to other proteins present in the sample. Typically a specific or selective reaction will be at least twice background signal or noise and more typically more than about 10 to 100 times background.

“Specifically hybridizable” and “complementary” as used herein, refer broadly to a nucleic acid can form hydrogen bond(s) with another nucleic acid sequence by either traditional Watson-Crick or other non-traditional types. The binding free energy for a nucleic acid molecule with its complementary sequence is sufficient to allow the relevant function of the nucleic acid to proceed, e.g., RNAi activity. Determination of binding free energies for nucleic acid molecules is well known in the art. See, e.g., Turner, et al. (1987) CSH Symp. Quant. Biol. LII: 123-33; Frier, et al. (1986) PNAS 83:9373-77; Turner, et al. (1987) J. Am. Chem. Soc. 109:3783-85. A percent complementarity indicates the percentage of contiguous residues in a nucleic acid molecule that can form hydrogen bonds (e.g., Watson-Crick base pairing) with a second nucleic acid sequence (e.g., about at least 5, 6, 7, 8, 9, 10 out of 10 being about at least 50%, 60%, 70%, 80%, 90%, and 100% complementary, inclusive). “Perfectly complementary” or 100% complementarity refers broadly all of the contiguous residues of a nucleic acid sequence hydrogen bonding with the same number of contiguous residues in a second nucleic acid sequence. “Substantial complementarity” refers to polynucleotide strands exhibiting about at least 90% complementarity, excluding regions of the polynucleotide strands, such as overhangs, that are selected so as to be noncomplementary. Specific binding requires a sufficient degree of complementarity to avoid non-specific binding of the oligomeric compound to non-target sequences under conditions in which specific binding is desired, i.e., under physiological conditions in the case of in vivo assays or therapeutic treatment, or in the case of in vitro assays, under conditions in which the assays are performed. The non-target sequences typically may differ by at least 5 nucleotides.

“Signs” of disease, as used herein, refers broadly to any abnormality indicative of disease, discoverable on examination of the patient; an objective indication of disease, in contrast to a symptom, which is a subjective indication of disease.

“Solid support,” “support,” and “substrate,” as used herein, refers broadly to any material that provides a solid or semi-solid structure with which another material can be attached including but not limited to smooth supports (e.g., metal, glass, plastic, silicon, and ceramic surfaces) as well as textured and porous materials. Exemplary solid supports include beads, such as activated beads, magnetically responsive beads, or fluorescently labeled beads.

“Subjects” as used herein, refers broadly to anyone suitable to be treated according to the presently disclosed inventions include, but are not limited to, avian and mammalian subjects, and are preferably mammalian. Mammals in the context of the presently disclosed inventions include, but are not limited to, canines, felines, bovines, caprines, equines, ovines, porcines, rodents (e.g., rats and mice), lagomorphs, primates, humans. Any mammalian subject in need of being treated according to the presently disclosed inventions is suitable. Human subjects of any gender and at any stage of development (i.e., neonate, infant, juvenile, adolescent, adult, elderly) can be treated according to the present invention. The present invention may also be carried out on animal subjects, particularly mammalian subjects such as mice, rats, dogs, cats, cattle, goats, sheep, and horses for veterinary purposes, and for drug screening and drug development purposes. “Subjects” is used interchangeably with “patients.” In preferred embodiments of the disclosed invention, the subject is a human.

“Symptoms” of disease as used herein, refers broadly to any morbid phenomenon or departure from the normal in structure, function, or sensation, experienced by the patient and indicative of disease.

“Therapy,” “therapeutic,” “treating,” or “treatment”, as used herein, refers broadly to treating a disease, arresting, or reducing the development of the disease or its clinical symptoms, and/or relieving the disease, causing regression of the disease or its clinical symptoms. Therapy encompasses prophylaxis, treatment, remedy, reduction, alleviation, and/or providing relief from a disease, signs, and/or symptoms of a disease. Therapy encompasses an alleviation of signs and/or symptoms in patients with ongoing disease signs and/or symptoms (e.g., tumor growth, metastasis). Therapy also encompasses “prophylaxis”. The term “reduced”, for purpose of therapy, refers broadly to the clinical significant reduction in signs and/or symptoms. Therapy includes treating relapses or recurrent signs and/or symptoms (e.g., tumor growth, metastasis). Therapy encompasses but is not limited to precluding the appearance of signs and/or symptoms anytime as well as reducing existing signs and/or symptoms and eliminating existing signs and/or symptoms. Therapy includes treating chronic disease (“maintenance”) and acute disease. For example, treatment includes treating or preventing relapses or the recurrence of signs and/or symptoms (e.g., tumor growth, metastasis).

“Variable region” or “VR,” as used herein, refers broadly to the domains within each pair of light and heavy chains in an antibody that are involved directly in binding the antibody to the antigen. Each heavy chain has at one end a variable domain (VH) followed by a number of constant domains. Each light chain has a variable domain (VL) at one end and a constant domain at its other end; the constant domain of the light chain is aligned with the first constant domain of the heavy chain, and the light chain variable domain is aligned with the variable domain of the heavy chain.

“Vector,” as used herein, refers broadly to a plasmid, cosmid, phagemid, phage DNA, or other DNA molecule which is able to replicate autonomously in a host cell, and which is characterized by one or a small number of restriction endonuclease recognition sites at which such DNA sequences may be cut in a determinable fashion without loss of an essential biological function of the vector, and into which DNA may be inserted in order to bring about its replication and cloning. The vector may further contain a marker suitable for use in the identification of cells transformed with the vector.

The techniques and procedures are generally performed according to conventional methods well known in the art and as described in various general and more specific references that are cited and discussed throughout the present specification. See, e.g., Sambrook, et al. (2001) Molec. Cloning: Lab. Manual [3^{rd} Ed] Cold Spring Harbor Laboratory Press. Standard techniques may be used for recombinant DNA, oligonucleotide synthesis, and tissue culture, and transformation (e.g., electroporation, lipofection). Enzymatic reactions and purification techniques may be performed according to manufacturer's specifications or as commonly accomplished in the art or as described herein. The nomenclatures utilized in connection with, and the laboratory procedures and techniques of, analytical chemistry, synthetic organic chemistry, and medicinal and pharmaceutical chemistry described herein are those well known and commonly used in the art. Standard techniques may be used for chemical syntheses, chemical analyses, pharmaceutical preparation, formulation, and delivery, and treatment of patients.

### EXAMPLES

The invention now being generally described, it will be more readily understood by reference to the following examples, which are included merely for purposes of illustration of certain aspects and embodiments of the present invention, and are not intended to limit the invention.

### Example 1: Assays Detecting Binding and Lysis of Hematologic Tumor Cell Lines by NEO-201

### Methods

### Tumor Cell Lines

The acute promyelocytic leukemia cell line [HL-60] (ATCC® CCL-240™) was obtained from American Type Culture Collection (Manassas, VA, USA). The cell line was used at low passage number, free of Mycoplasma and cultured at 37° C./5% CO₂ in medium designated by the provider for propagation and maintenance.

### Human Healthy Donor PBMCs

Peripheral blood mononuclear cells (PBMCs) from human healthy donors were obtained from the National Institutes of Health Clinical Center Blood Bank (NCT00001846) under the appropriate Institutional Review Board approval and informed consent.

PBMCs were incubated overnight at 37° C./5% CO₂ in complete medium prior to be used as effector cells in the ADCC assay. The complete medium is composed by RPMI-1640 medium (Corning Life Science, Manassas, VA, USA), containing 10% fetal bovine serum 10% USA-sourced and heat-inactivated HyClone fetal bovine serum defined (GE Healthcare Life Sciences, Issaquah, WA, USA), 100 U/ml penicillin, 100 μg/mL streptomycin (Corning Life Science, Manassas, VA, USA).

### NK Cell Purification

NK effector cells were isolated from peripheral blood mononuclear cells (PBMCs) using Miltenyi Biotech Human NK Cell Isolation Kit (Miltenyi Biotech, Cologne, Germany) according to the manufacturer's protocol. NK cells isolated from healthy donors were incubated overnight at 37° C./5% CO₂ in complete medium prior to use as effector cells in the ADCC assay.

### Antibodies

The monoclonal antibody NEO-201 employed in this study was provided by Precision Biologics, Inc. The human IgG1 isotype control antibody was purchased by Thermo Fisher Scientific, Waltham, MA, USA.

### Flow Cytometry

Analysis of the expression of cell-surface antigen recognized by NEO-201 in HL-60 cell line was performed by flow cytometry. Cells were harvested and counted to obtain a concentration of (1.0×10⁶). Cells were then centrifuged, washed twice with cold PBS, and then stained with 10 μg/ml of FITC-conjugated NEO-201 antibody in 1×PBS+1% BSA (Teknova, Hollister, CA, USA) for 30 minutes at 4° C.

After staining, cells were washed twice with cold PBS and examined using the FACS CANTO II flow cytometer (BD, San Jose, CA, USA). Analysis of cellular fluorescence was performed using the FCS Express software. Positivity was determined confronting unstained cells with cells stained with FITC-conjugated NEO-201 antibody. Staining values >10% positive were considered positive for NEO-201 expression.

### In Vitro ADCC Assay

A 4-h ¹¹¹In-release assay was used to evaluate the ADCC mediated by NEO-201. The acute promyelocytic leukemia cell line HL-60 was used as target and PBMCs or isolated NK cells were used as effectors in presence of NEO-201 or lgG1 isotype control antibody or alone. Target cells were harvested and counted. 1×10⁶ target cells were suspended in complete medium, labeled with 20 μCi ¹¹¹In-oxyquinoline (GE Healthcare, Silver Spring, MD) and incubated at 37° C./5% CO₂ for 20 minutes. After incubation, cells were washed twice with complete medium and then seeded at 3000 cells/well in a 96-well round-bottom culture plate. Subsequently, NEO-201 or lgG1 isotype control antibody was added to target cells at a concentration of 10 μg/mL. Then, effectors were added to the plate. For the ADCC assay performed with PBMCs as effectors, PBMCs were added in the 96-well round-bottom culture plate at effector cell: target cell (E:T) ratios of 50:1 and 25:1. For the ADCC assay performed with NK cells as effectors, NK cells were added in the 96-well round-bottom culture plate at effector cell:target cell (E:T) ratios of 20:1 and 10:1.

Target cells were also incubated with complete medium alone to calculate the spontaneous release or with 0.05% Triton X-100 to calculate the complete lysis.

Then the plate was incubated at 37° C./5% CO₂ for 4 h.

After incubation, medium from wells in the plate was collected and the spontaneous release, complete lysis and specific lysis was counted at the Gamma Counter. Percentage of specific ADCC lysis was determined using the following equation:

Percent specific lysis=(experimental lysis−spontaneous release)/(complete lysis−spontaneous release)×100.

### Example 2: Detection of NEO-201 Expression by Hematological Malignancy Cells

The ability of NEO-201 to bind hematological neoplasms was analyzed by flow cytometry. Human HL-60 cells (acute promyelocytic leukemia cell line) were used as the target.

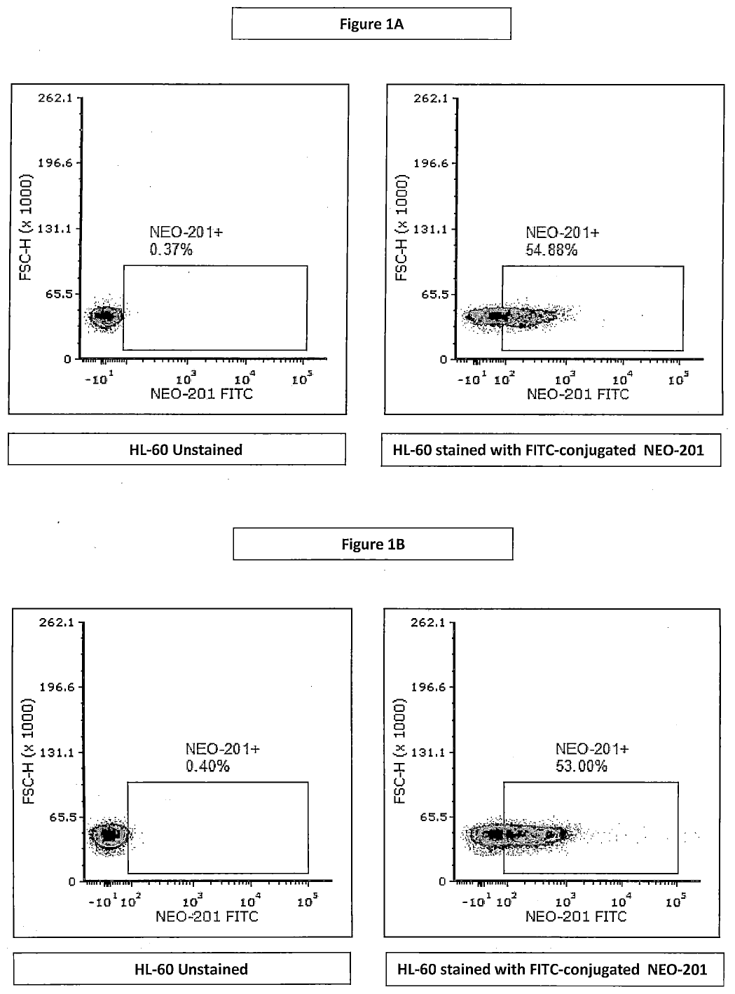

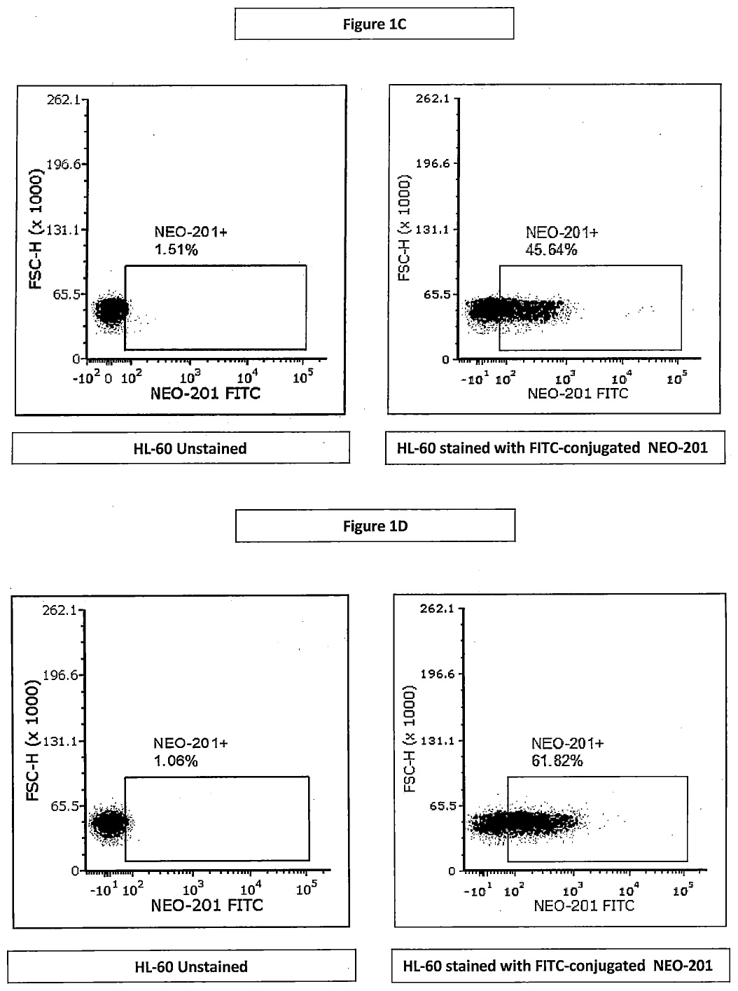

Data are presented as percentage of cells expressing the antigen recognized by NEO-201. Reactivity with NEO-201 was determined by comparing unstained cells (FIG. 1A-1D, left panels) with cell stained with 10 μg/mL FITC-conjugated NEO-201 (FIG. 1, right panels). Positivity was defined as % of positive cells ≥10%. Results are shown for four independent replicates (FIGS. 1A-1D, respectively).

The results of two independent experiments show that 54.88%, 53.00%, 45.64%, and 61.82% of HL-60 cells scored as NEO-201 positive (FIGS. 1A-1D, right panels, respectively), compared to 0.37%, 0.40%, 1.51%, and 1.06% for controls (unstained HL-60 cells, FIGS. 1A-1D, left panels).

### Example 3: NEO-201 can Kill Hematological Malignancy Cells In Vitro

The HL-60 cell line was used as a target to evaluate the ADCC mediated by NEO-201 in an in vitro ADCC assays using PBMCs or purified NK cells as effector cells. Cells were incubated with 10 μg/mL of NEO-201 or human lgG1 (negative control).

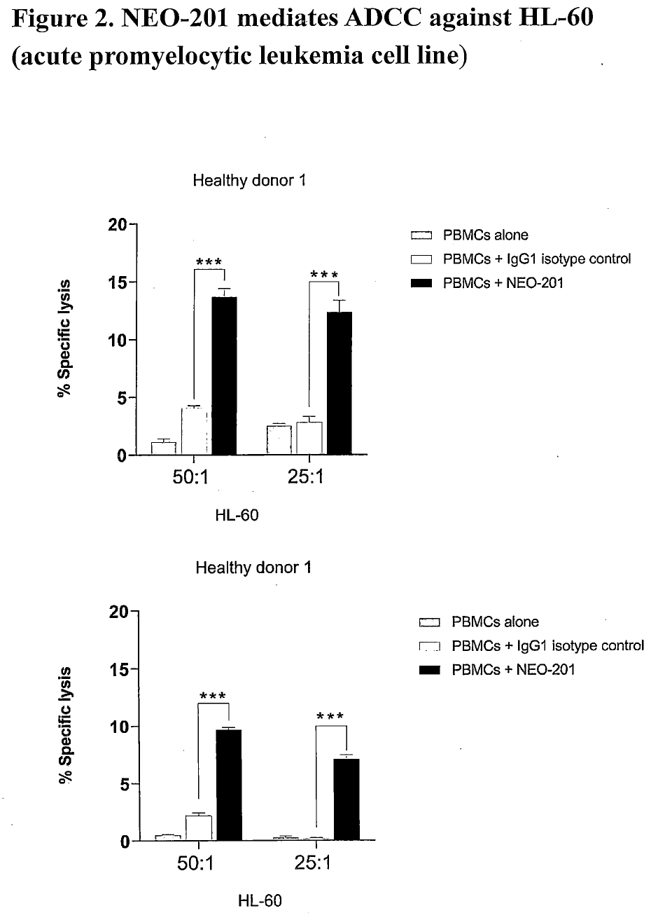

PBMCs from one healthy donor were used as effector cells at E:T ratios of 50:1 or 25:1 in two independent experiments as indicated. Results are presented as mean of percentage specific lysis±SD (Standard deviation) from 3 replicate wells in each experiment. The results of the in vitro cytotoxicity assays using PBMCs as effector cells are shown in Table 1 below and are depicted graphically in FIG. 2.

TABLE 1
Results of in vitro cytotoxicity assays
using PBMCs as effector cells.

| HL-60 (acute promyelocytic | | % specific lysis (SD) | | | |
|---|---|---|---|---|---|
| leukemia cell line) | | (Healthy Donor 1) | | | |
| Antibody | E:T Ratio | Experiment 1 | | Experiment 2 | |
| PBMCs alone | 50:1 | 1.14 | (0.47) | 0.49 | (0.06) |
| | 25:1 | 2.56 | (0.29) | 0.29 | (0.14) |
| IgG1 + | 50:1 | 4.07 | (0.34) | 2.18 | (0.40) |
| PBMCs | 25:1 | 2.87 | (0.81) | 0.15 | (0.14) |
| NEO-201 + | 50:1 | 13.67 | (1.31)* | 9.67 | (0.38)* |
| PBMCs | 25:1 | 12.35 | (1.77)^{&} | 7.10 | (0.62)^{&} |

*statistically significant (p < 0.001) by 2way ANOVA (NEO-201 + PBMCs vs IgG1 + PBMCs at 50:1 E:T ratio).
^{&}statistically significant (p < 0.001) by 2way ANOVA (NEO-201 + PBMCs vs IgG1 + PBMCs at 25:1 E:T ratio).

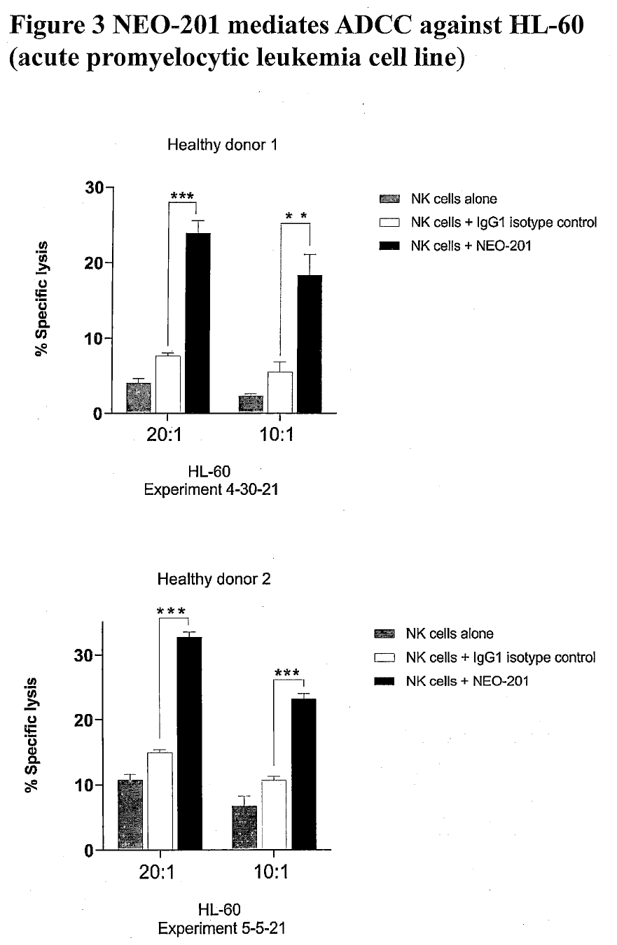

Cytotoxicity assays were also performed using NK cells as effector cells. The cells were incubated with 10 μg/mL of NEO-201 or human lgG1 (negative control). NK cells from two healthy donors were used as effector cells at the indicated E:T ratios in two independent experiments. Results are presented as mean of % specific lysis±SD (Standard deviation) from 3 replicate wells in each experiment. The results of the in vitro cytotoxicity assays using purified NK cells as effector cells are shown in Table 2 below and are depicted graphically in FIG. 3.

TABLE 2
Results of in vitro cytotoxicity assays using
purified NK cells as effector cells.

| HL-60 (acute promyelocytic | | % specific lysis (SD) | | | |
|---|---|---|---|---|---|
| leukemia cell line) | | Experiment 1 | | Experiment 2 | |
| | E:T | (Healthy | | (Healthy | |
| Antibody | Ratio | Donor 1) | | Donor 2) | |
| NK cells alone | 20:1 | 4.06 | (0.99) | 10.75 | (1.54) |
| | 10:1 | 2.27 | (0.48) | 6.69 | (2.64) |
| IgG1 + NK | 20:1 | 7.67 | (0.54) | 14.98 | (0.71) |
| cells | 10:1 | 5.47 | (1.55) | 10.66 | (1.04) |
| NEO-201 + NK | 20:1 | 23.90 | (2.84)* | 32.67 | (1.31)* |
| cells | 10:1 | 18.36 | (4.71)^{#} | 23.15 | (1.58)^{&} |

*statistically significant (p < 0.001) by 2way ANOVA (NEO-201 + NK cells vs IgG1 + NK cells at 20:1 E:T ratio).
^{#}statistically significant (p < 0.01) by 2way ANOVA (NEO-201 + PBMCs vs IgG1 + PBMCs at 10:1 E:T ratio).
^{&}statistically significant (p < 0.001) by 2way ANOVA (NEO-201 + PBMCs vs IgG1 + PBMCs at 10:1 E:T ratio).

The results show that NEO-201 effectively killed HL-60 cells in vitro with PBMCs or purified NK cells as effector cells.

### Example 4: Binding of NEO-201 to Hematological Neoplastic Cell Lines In Vitro

### Background

We previously demonstrated that, in addition to solid tumors (Fantini M, et al. “Preclinical Characterization of a Novel Monoclonal Antibody NEO-201 for the Treatment of Human Carcinomas”, Front Immunol. 2017; 8:1899-3); Fantini M, et al., “An IL-15 Superagonist, ALT-803, Enhances Antibody-Dependent Cell-Mediated Cytotoxicity Elicited by the Monoclonal Antibody NEO-201 Against Human Carcinoma Cells”, Cancer Biother Radiopharm. 2019; 34 (3): 147-59; Zeligs K P, et al., “Evaluation of the Anti-Tumor Activity of the Humanized Monoclonal Antibody NEO-201 in Preclinical Models of Ovarian Cancer”, Front Oncol. 2020; 10:805); the antigen recognized by NEO-201 is also expressed on specific subsets of human hematopoietic cells (Fantini M, et al., “The Monoclonal Antibody NEO-201 Enhances Natural Killer Cell Cytotoxicity Against Tumor Cells Through Blockade of the Inhibitory CEACAM5/CEACAM1 Immune Checkpoint Pathway”, Cancer Biother Radiopharm. 2020; 35 (3): 190-8). In this regard, we observed that 98.9% of CD15+ granulocytes and about 4.6% of CD4⁺ T cells were positive for NEO-201 staining. Conversely, NEO-201 did not bind to B cells, NK cells, monocytes, CD8+ T cells and a majority of CD4⁺ T cells (Fantini M, et al., “The Monoclonal Antibody NEO-201 Enhances Natural Killer Cell Cytotoxicity Against Tumor Cells Through Blockade of the Inhibitory CEACAM5/CEACAM1 Immune Checkpoint Pathway”, Cancer Biother Radiopharm. 2020; 35 (3): 190-8).

Starting from these observations, we evaluated the reactivity of NEO-201 against hematological neoplastic cell lines in vitro, such as Acute Myeloid Leukemia (AML), Multiple Myeloma (MM), Acute Lymphoblastic Leukemia (ALL), Mantel Cell Lymphoma (MCL) cells.

### Materials and Methods

### Cell Lines

Cell lines used were six AML (HL60, U937, MOLM13, AML2, IMS-M2 and OCL-AML3), two MM (OPM2, MM1.S), two ALL (SUP-B15, RPMI8402) and four MCL (Jeko-1, Z138, JVM2 and JVM13).

Cell lines were used at low passage number, free of Mycoplasma and cultured at 37° C./5% CO₂ in medium designated by the provider for propagation and maintenance.

### Flow Cytometry

Analysis of the expression of cell-surface antigen recognized by NEO-201 in hematological neoplastic cell lines was performed by flow cytometry. Cells were harvested and counted to obtain a concentration of (1.0×10⁶). Cells were then centrifuged, washed twice with cold PBS, and then stained in 1×PBS+1% BSA for 20 minutes in the dark at room temperature with the following monoclonal antibodies: CD15, CD45, CD38, CD138, CD14, CD19 and NEO-201.

After staining, cells were washed twice with cold PBS and examined using Navios flow cytometer. Analysis of cellular fluorescence was performed using the Kaluza software (Beckman Coulter). Positivity was determined by using fluorescence minus one controls. Staining values ≥10% positive for NEO-201 were considered positive for expression of the antigen recognized by NEO-201.

### Results

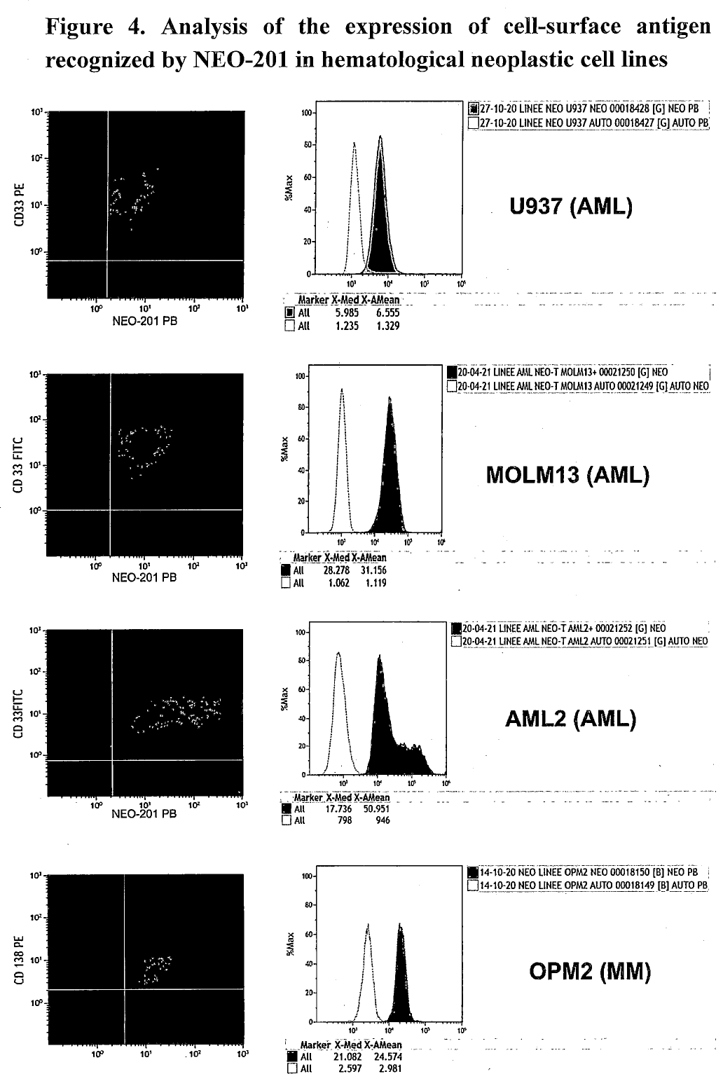

NEO-201 was found to react with AML and MM cell lines. 5 of 6 AML cell lines tested bound to NEO-201 and the % of positive cells were 47%, 99.5%, 100%, 100% and 97.8% for HL60, U937, MOLM13, AML2 and IMS-M2, respectively. The % of positive cells in the two MM cell line were 99% and 18% for OPM2 and MM1.S, respectively. NEO-201 did not react against the two ALL and the four MCL cell lines tested (Table 3 and FIG. 4).

FIG. 4 contains an analysis of the expression of cell-surface antigen recognized by NEO-201 in hematological neoplastic cell lines Data are presented as percentage of cells expressing the antigen recognized by NEO-201. Positivity was determined by using fluorescence minus one controls. Staining values ≥10% positive for NEO-201 staining were considered positive for expression of the antigen recognized by NEO-201.

Table 3 provides a summary of the analysis of the expression of cell-surface antigen recognized by NEO-201 in hematological neoplastic cell lines. NEO-201 positive cell lines appear in bold text and NEO-201 positivity was defined as % positive ≥10%.

TABLE 3
Expression of cell-surface antigen recognized by
NEO-201 in hematological neoplastic cell lines

| | Tumor | % | | | Tumor | % | |
|---|---|---|---|---|---|---|---|
| Cell line | Type | positive | MFI | Cell line | Type | positive | MFI |
| HL60 | AML | 47.0 | 4,864 | SUP-B15 | ALL | Neg | NA |
| U937 | AML | 99.5 | 5,985 | RPMI-8402 | ALL | Neg | NA |
| MOLM13 | AML | 100 | 28,278 | Jeko-1 | MCL | Neg | NA |
| AML2 | AML | 100 | 17,736 | Z138 | MCL | Neg | NA |
| IMS-M2 | AML | 97.8 | 4,640 | JVM2 | MCL | Neg | NA |
| OCL-AML3 | AML | Neg | NA | JVM13 | MCL | Neg | NA |
| OPM2 | MM | 99.0 | 21,082 | | | | |
| MM1.S | MM | 18.0 | 2,380 | | | | |

### Example 5: Identification of mAb NEO-201 Binding O-Glycan Epitopes

### Introduction

The aberrant O-glycans expression at the cancer cell surface occur as saccharide components of membrane-bound N-acetyl galactosamine (O-GalNAc) glycoproteins (T and Tn antigen) and glycolipids (Lewis a and Lewis x). The sialylation of the sugar of the glycan chain introduces an additional diversity in the O-glycan repertoire expressed by cancer cells. Glycosylation is an important post-translation modification of proteins and lipids and is strongly affected by oncogenesis. All these aberrant O-glycans may serve as potential targets to improve the diagnosis and treatment of tumors and provide the molecular probes for their specific recognition. Monoclonal antibodies that specifically recognize the Tn and T antigens have been widely used to detect malignant cells.

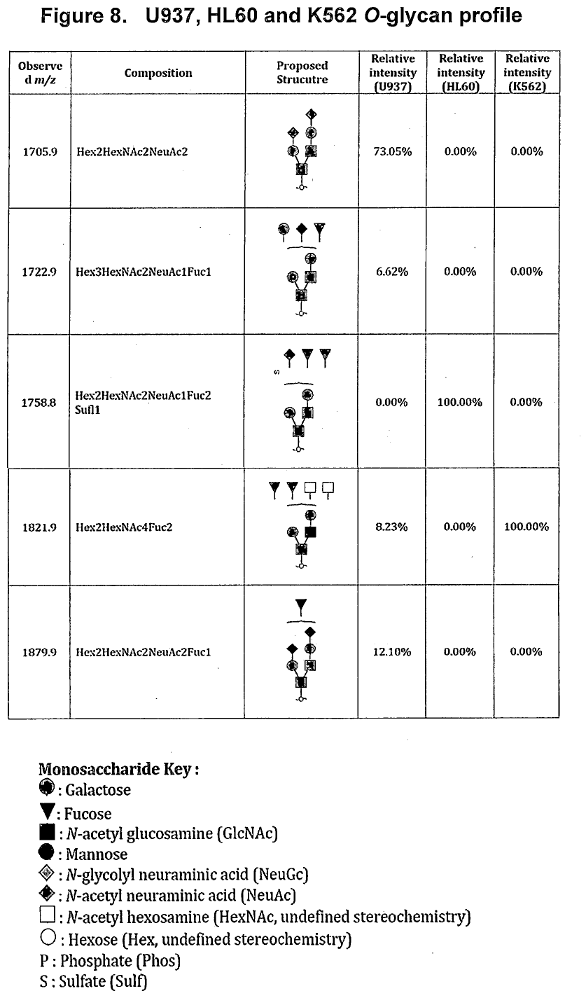

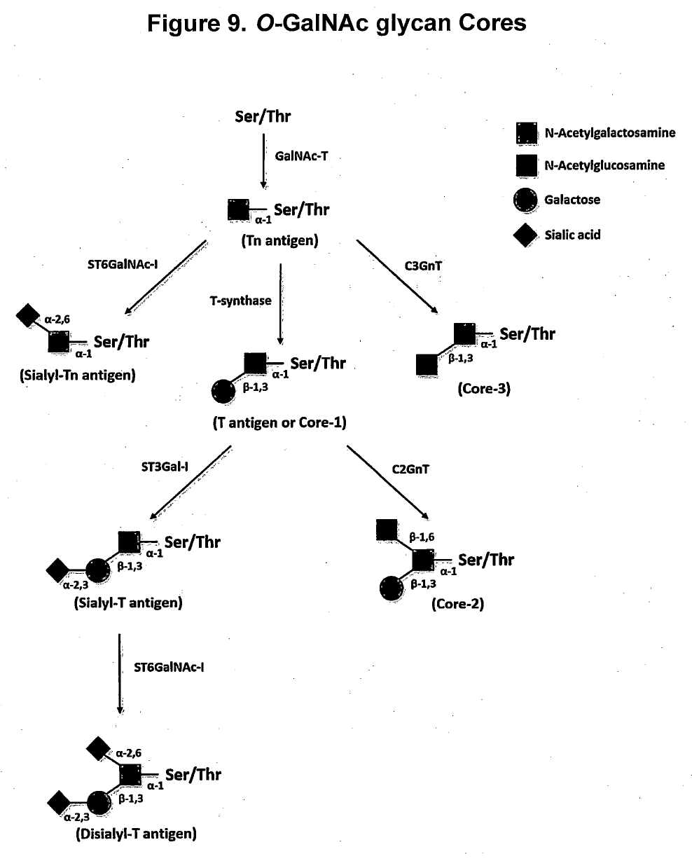

The O-glycosylation pathway starts with the addition of a single N-acetyl galactosamine (GalNAc) to a serine or threonine residue, thus forming the Tn antigen epitope. The Tn antigen can be further elongated with galactose to form the T antigen, also known as Core 1 (Thomsen-Friedenreich antigen) or with N-acetylglucosamine (GlcNAc) to form Core 3 (FIG. 9). Core 1 O-glycans in healthy cells are generally further extended to complex branched Core 2 with GlcNAc or Tn with two GLcNAc to form Core 4 structure. Core 3 and Core 4 structures are exclusively present in the intestinal tract. Core structure 5-8 have an extremely restricted occurrence (FIG. 9 and FIG. 8). Mucins with Core 5 have been reported in human meconium and intestinal adenocarcinoma tissue, whereas Core 6 structures are found in human intestinal mucin and ovarian cyst mucin. Core 8 has been reported in human respiratory mucin. Bovine submaxillary mucin was shown to contain a core 7 structure. FIG. 8 shows the structures of O-glycan Cores found in mucins. Tumor cells are characterized by the expression of truncated O-glycans, such as Tn antigen and T antigen, on the cell surface. These truncated structures can be sialylated forming sialyl-Tn (sTn) and sialyl- of disialyl-T (ST), which prevents further elongation of the glycan structure. Cores 1-4 have been shown to occur as extended complex O-glycans. The Tn and T antigens and their sialylated structures, sTn and ST antigens are expressed by multiple tumor types, especially those of epithelial origin such as breast cancers, ovarian cancers, gastric cancers, and colon cancers. Truncated O-glycans, in general support tumor progression and their presence is strongly correlated to poor prognosis.

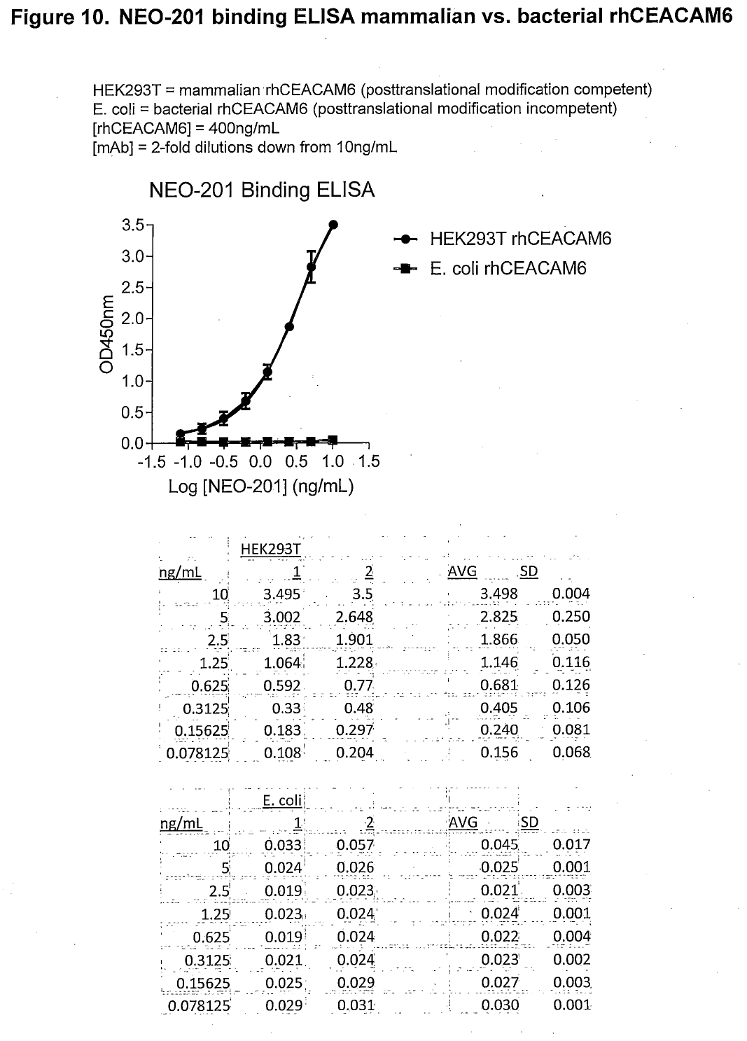

NEO-201 is an IgG1 mAb reactive against many different human carcinomas expressing NEO-201 target antigen, but not against most normal epithelial tissues. NEO-201 can mediate antitumor activity against tumor cells through multiple mechanisms such as antibody-dependent cellular cytotoxicity (ADCC), complement dependent cytotoxicity (CDC), and blockade of the CEACAM5/CEACAM1 immune checkpoint inhibitory pathway. In addition to solid tumors, we show herein that the NEO-201 target is also found on human hematopoietic cells. Flow cytometry analysis has demonstrated that 98.9% of CD15+ granulocytes and about 4.6% of CD4+ T cells were positive for NEO-201 staining. No binding was observed with NEO-201 with respect to B cells, NK cells, monocytes, CD8+ T cells and a majority of CD4+ T cells. We have also demonstrated that NEO-201 binds to mammalian expressed rhCEACAM6 but not bacterial expressed rhCEACMA6 (FIG. 10). Based on these observations, we evaluated whether NEO-201 potentially would be reactive against hematological neoplastic cell lines in vitro, such as Acute Myeloid Leukemia (AML), Multiple Myeloma (MM), Acute Lymphoblastic Leukemia (ALL), and Mantel Cell Lymphoma (MCL) cell lines.

### Materials and Methods

### Identification of O-Glycans Bind to NEO-201 Using O-Glycan Arrays

In the experiments in FIG. 11, the glycan array was blocked for 30 min using our Glycan Array Blocking Buffer. It was then washed 3× using our Glycan Array Assay Buffer (GAAB) (which is TBS-T based). The samples were diluted in GAAB to the desired concentrations, and then applied directly to the glycan array. The array was covered and shaken at 80 rpm for 1 hour at RT. The array was then washed 3× again with GAAB, and then detection antibody was diluted in GAAB and applied to the array. It was covered from light and shaken at 80 rpm for 1 hour at RT, and then washed 3× with GAAB and 2× with MilliQ water. It was read using an Innopsys InnoScan 710 Microarray Scanner with a high-power laser at 5PMT. Software was used to detect each spot on the array and calculate the RFU intensity for each spot. Background RFU was subtracted from each spot's RFU value. The median of each glycan's spot was determined and graphed.

### O-Glycan Array Layout:

PC1: B1-PEG-NH2; PC2: human lgG; PC3: mouse lgG; PC4: Rabbit lgG

NEO-201 was used at three concentrations (100 μg/ml, 20 μg/ml and 4 μg/ml) to incubate with the O-glycan Array for 1 hour at RT. The Arrays were then washed and incubated with anti-human lgG Fc Cy3 at a concentration of 20 μg/ml for 1 hour at RT. The Arrays were washed 3× with GAAB and 2× with MilliQ water. It was read using an Innopsys InnoScan 710 Microarray Scanner with a high-power laser at 5PMT. Software was used to detect each spot on the array and calculate the RFU intensity for each spot. Background RFU was subtracted from each spot's RFU value.

### Elucidate O-Glycan Profiles of Tumor Cell Lines and Estimate the Relative Abundance of Each Glycan Detected

Glassware and glass tubes are cleaned with Milli Q water and dried beforehand.

Reagents are weighted on aluminum foils with utensils cleaned with Milli Q water beforehand. Whenever possible, liquid reagents are handled with disposable glass pipettes. Solvents are HPLC grade or higher.

### Removal of N-Glycans:

1. After 3 washes in PBS the cell pellet was resuspended in 1 ml of lysis buffer (25 mM TRIS, 150 mM NaCl, 5 mM EDTA, 0.5% w/v CHAPS, pH 7.4) and sonicated (5 pulses of 10 s). The lysed sample was next dialyzed against 50 mM ammonium bicarbonate for 24 h at 4° C. and with the dialysis buffer changed three times.
2. After lyophilization, the dialyzed material was resuspended in 1 ml of a 2 mg/ml DTT (1,4-Dithiothreitol) solution and incubated at 50° C. for 90 mins.
3. Prepare a fresh 12 mg/ml solution of iodoacetamide in 0.6 M TRIS buffer pH 8.5. Add 0.5 ml of IAA solution to the DTT-treated sample and incubate at RT in the dark for 90 mins.
4. Dialyze the sample against 50 mM ammonium bicarbonate at 4° C. for 16 h-24 h, changing the buffer 3 times. The molecular cut-off should be between 1 and 5 kDa. After dialysis transfer the sample into a 15 ml tubes and lyophilize the sample.
5. Resuspend the dry sample in 0.5 ml of a 50 μg/ml solution of TPCK-treated trypsin in 50 mM ammonium bicarbonate and incubate overnight (12-16 h) at 37° C. Stop the reaction by adding 2 drops of 5% acetic acid.
6. Condition a C18 Spe-Pak (50 mg) column with methanol, 5% acetic acid, 1-propanol and 5% acetic acid. Load the trypsin-digested samples onto the C18 column.
7. Wash the column with 4 ml of 5% acetic acid and elute the peptides from the C18 column with 2 ml of 20% 1-propanol, then 2 ml 40% 1-propanol and finally 2 ml of 100% 1-propanol. Pooled all the eluted fractions and lyophilized the sample.
8. Resuspend thoughtfully the dried material in 200 μl of 50 mM ammonium bicarbonate and add 2 μl of PNGaseF Incubate at 37° C. for 4 h. Then add another 3 μl of PNGaseF for overnight (12-16 h) incubation at 37° C. Stop the reaction by adding 2 drops of 5% acetic acid.
9. Condition a C18 Spe-Pak (50 mg) column with Methanol, 5% acetic acid, 1-Propanol and 5% acetic acid. Load the PNGaseF-digested sample onto the C18 column. Discard the flow through.

### Preparation of O-Glycans:

1. The material bound to the column (containing peptides and potentially O-glycopeptides) is collected by sequential elutions with 1 ml of 20% 1-propanol, 1 ml of 40% 1-propanol and 1 ml of 100% 1-propanol. Lyophilize the eluted fractions.
2. Add 400 μl of a 55 mg/ml NaBH₄ (sodium borohydride) in 0.1 M NaOH solution to the sample. Incubate at 45° C. overnight.
3. Terminate the reaction by adding 3-4 drops of pure (100%) acetic acid until fizzing stops.
4. Prepare and condition a Dowex (50W X8, mesh size 200-400) ion exchange resin column by washing the column with 10 ml of 5% acetic acid.
5. Load the acetic acid-neutralized sample onto the column and wash with 3 ml of 5% acetic acid. Collect and pool flow through and wash. Lyophilize the collected material.
6. Add 1 ml of acetic acid:methanol solution (1:9; v/v=10%) to the lyophilized sample. Vortex thoroughly and dry under a stream of nitrogen. Repeat this co-evaporation step three more times.
7. Condition a C18 Column
8. Resuspend the co-evaporated dried sample in 200 μl of 50% methanol and load onto the conditioned C18 column. Wash the column with 4 ml of 5% acetic acid. Collect and pool flow through and wash, lyophilize the sample and proceed to permethylation.

### Permethylation:

The Preparation of the slurry NaOH/DMSO solution is made fresh every time. Mortar, pestle, and glass tubes are washed with Milli Q water and dried beforehand. Whenever possible, liquid reagents are handled with disposable glass pipettes. Solvents are HPLC grade or higher.
1. With a clean and dry mortar and pestle grind 7 pellets of NaOH in 3 ml of DMSO. Add 1 ml of this slurry solution to dry sample in a glass tube with a screw cap.
2. Add 500 μl of Iodomethane, close the lid tightly and shake the sample at RT for ˜30 min. The mixture should turn white and even become solid as it reaches completion.
3. Open slowly the cap as gas pressure has built up. Add 1 ml of MilliQ water to stop the reaction, vortex the tube until all solid is dissolved.
4. Add 1 ml of Chloroform and additional 3 ml of MilliQ water, vortex thoroughly the tube to mix both phases and centrifuge briefly to separate the chloroform and the water phases (˜5000 rpm, <20 sec).
5. Discard the aqueous top layer and repeat the wash 2 more times adding 3 ml of Milli Q water.
6. Dry the chloroform fraction with a SpeedVac (˜20-30 min).
7. Condition a C18 Spe-Pak (200 mg) column with Methanol, MiliQ water, Acetonitrile and MilliQ water. Resuspend the dry sample with 200 μl of 50% methanol and load onto the column. Wash the tube with 1 ml of 15% acetonitrile and load onto the column. Wash the column with 2 ml of 15% acetonitrile.
8. Elute the column in a clean glass tube with 3 ml of 50% acetonitrile. Lyophilized the eluted fraction for MS analysis.

### MS Maldi Analysis:

MS data was acquired on a Bruker UltraFlex II MAL DI-TOF Mass Spectrometer instrument. Reflective positive mode is used, and data is usually recorded between 500 m/z and 4000 m/z for O-glycans.

For each MS O-glycan profiles the aggregation of 20,000 laser shots or more were considered for data extraction. Mass signals of a signal/noise ratio of at least 2 were considered and only MS signals matching an O-glycan composition were considered for further analysis and annotated. Subsequent MS post-data acquisition analysis were made using mMass (Strohalm, M., Kavan, D., Novak, P., Volny, M., and Havlicek, V. (2010), “mMass 3: a cross-platform software environment for precise analysis of mass spectrometric data”, Anal Chem 82, 4648-4651).

Tumor Cell Lines and Human Neutrophils Used in this Analysis:

NEO-201 antigens positive cell lines (CFPAC-1: pancreatic cancer, human neutrophils from a normal donor, HL-60: AML, U937: AML and K562: CML).

### Identification of the NEO-201 Binding Region in CEACAM6 and CEACAM5 C-Terminus

For the expression of CEACAM6 and CEACAM5, PCR fragment of CEACAM6 and CEACAM5 were built in DHFR vector via restriction sites Nhe1 and Hind111. CEACAM6 cDNA was obtained by RT-PCR with CFPAC-1 mRNA extract as template and CEACAM5 cDNA was brought from ATCC (#MGC-34212). The sequences were verified by primers BGH-REV (CTAGAAGGCACAGTCGAGGC) and CMV- for (CGCAAATGGGCGGTAGGCGTG). 4 μg plasmid was transiently transfected into 1×10⁶ mammalian HEK293T cells (90% confluency) seeding in 6-well plate and cultured for 48-72 hours.

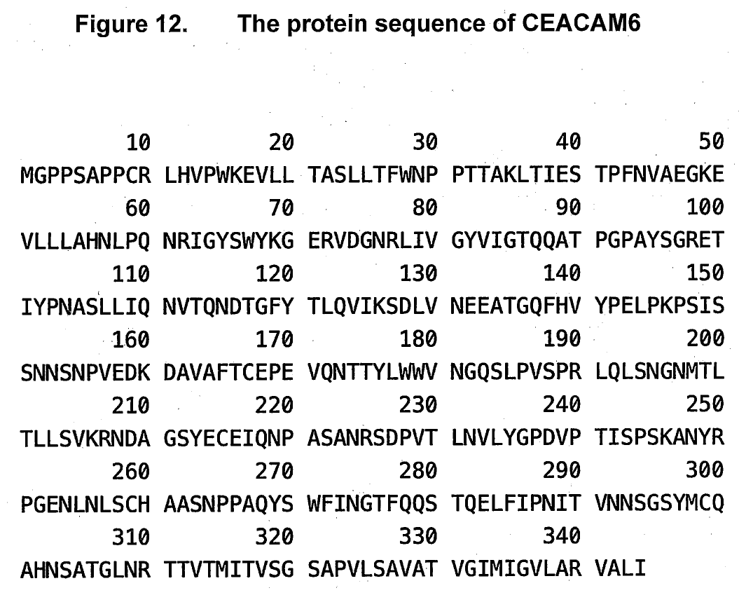

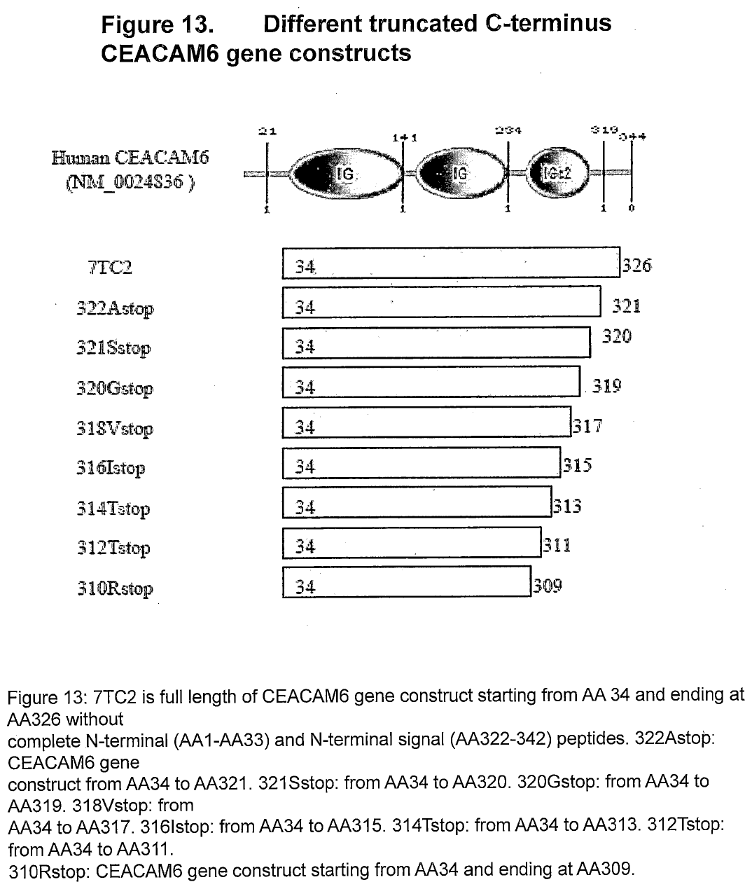

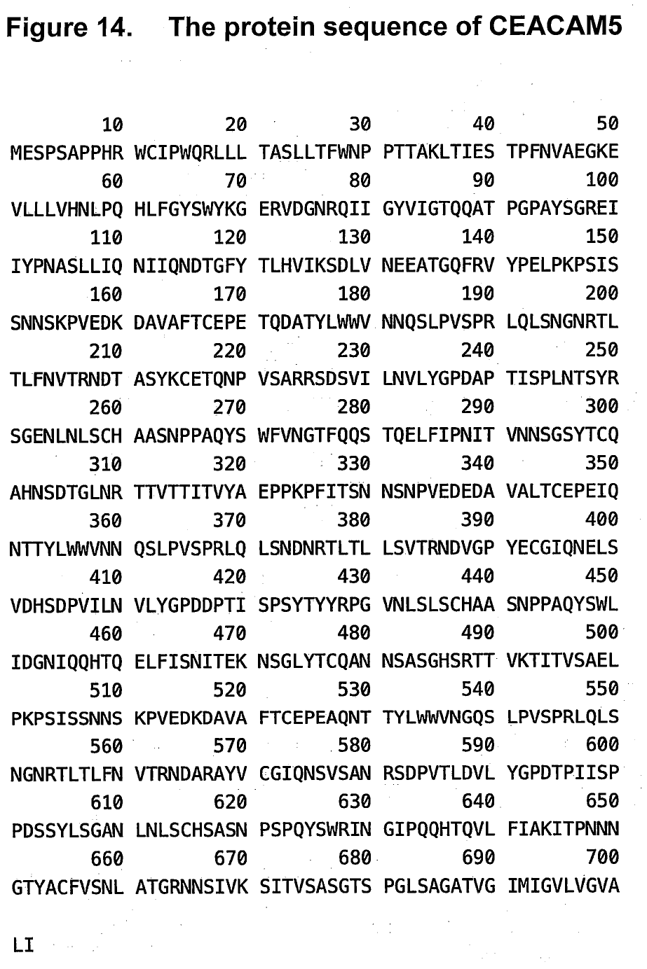

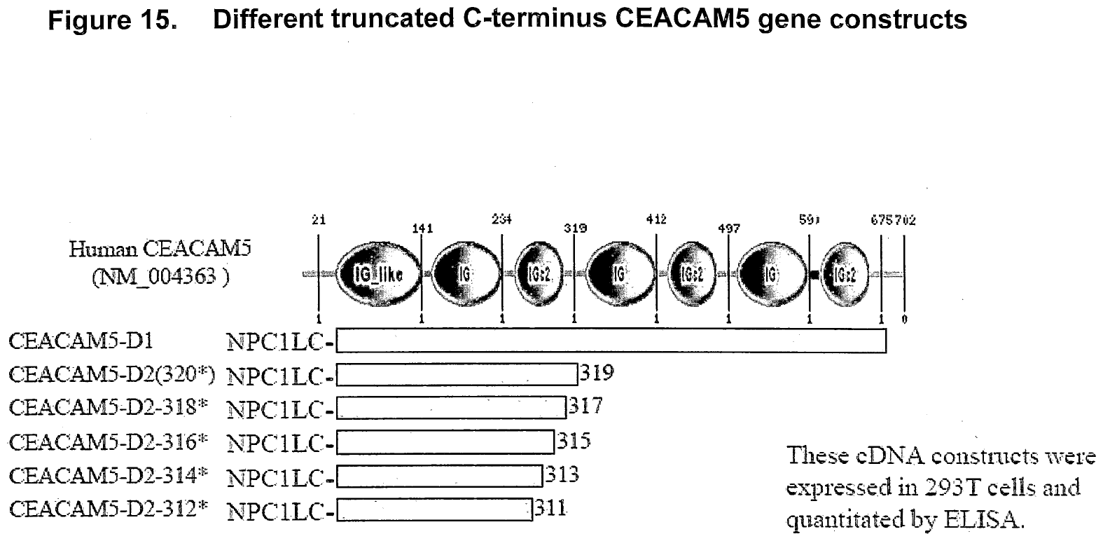

FIG. 12 shows the amino acid sequence of CEACAM6. Different truncated C-terminus CEACAM6 gene constructs were designed as illustrated in FIG. 13. The truncation study is to identify the NEO-201 binding area of C-terminus of CEACAM6. FIG. 14 shows the amino acid sequence of CEACAM5. Different truncated C-terminus CEACAM5 gene constructs were designed as illustrated in FIG. 15. The procedure for the expression of the truncated C-terminus constructs was the same as to the method described above. All deletions were transfected into HEK293T cells, harvest the cell pellet and/or supernatant after 48-72 hours. Supernatant binding activity was quantitated by ELISA. The truncation study is to identify the NEO-201 binding area of C-terminus of CEACAM5.

NEO-201 Binds to Mammalian Expressed rhCEACAM6 but not Bacterial Expressed rhCEACMA6

DNA plasmids comprising CEACAM6 coding sequences, as described above were expressed in bacterial (E. coli BL21) and mammalian HEK293T cells. The expressed proteins were tested for their ability to be detected by NEO-201 mAb in ELISA to ascertain that the binding epitope(s) of NEO-201 comprises carbohydrates.

Briefly, ninety-six-well plates were first coated overnight at 4° C. with 100 μL/well of 400 ng/ml recombinant human CEACAM6 expressed in E. coli BL21 or in HEK293T cells. Plates were then washed with 1× Tris-buffered saline (TBS)+0.05% Tween-20 and then blocked with 200 μL/well of 5% milk in 1× TBS for 1 hr at 37° C. Plates were then washed, and then 100 μl/well of NEO-201 antibody was added in 2-fold serial dilution from 10 ng/ml to 0.078 ng/ml and incubated for 1 hr at 37° C. Plates were washed, and 100 μL/well of anti-human IgG antibody peroxidase conjugate was added to the plate and incubated for 1 hr at 37° C. Plates were washed, and 100 μL/well of tetramethylbenzidine (TMB) substrate solution was added at RT in the dark. The reaction was stopped by adding H2SO4, and absorption at 450 nm was read using a plate reader.

### Flow Cytometry of NEO-201 Binding to Human Carcinoma Cell Lines

The chronic myeloid leukemia cell line [K562] (ATCC® CCL-243™) and the pancreatic cell line [CFPAC-1] (ATCC® CRL-1918™) were obtained from American Type Culture Collection (Manassas, VA, USA). Cell lines were used at low passage number, free of Mycoplasma and cultured at 37° C./5% CO₂ in medium designated by the provider for propagation and maintenance.

Analysis of the expression of cell-surface antigen recognized by NEO-201 was performed by flow cytometry. Cells were harvested and counted to obtain a concentration of (1.0×10⁶). Cells were then centrifuged, washed twice with cold PBS, and then stained with 10 μg/ml of Pacific Blue-conjugated NEO-201 antibody in 1×PBS+1% BSA (Teknova, Hollister, CA, USA) for 30 minutes at 4° C. After staining, cells were washed twice with cold PBS and examined using the FACSVerse flow cytometer (BD, San Jose, CA, USA). Analysis of cellular fluorescence was performed using the BD FACSuite software. Positivity was determined confronting unstained cells with cells stained with Pacific Blue-conjugated NEO-201 antibody. Staining values >10% positive were considered positive for NEO-201 antigen expression.

### Results

NEO-201 Binds to Mammalian Expressed rhCEACAM6 but not Bacterial Expressed rhCEACMA6

As shown in FIG. 10 ELISA results revealed that NEO-201 binds to mammalian expressed rhCEACAM6 but does not bind to bacterial expressed rhCEACMA6.

### Binding Affinity of NEO-201 to the O-Glycan Array

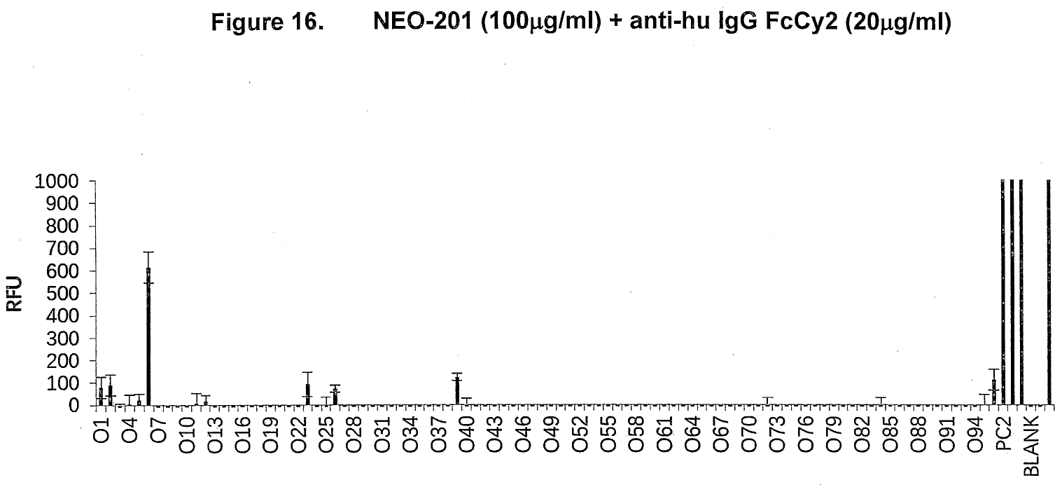

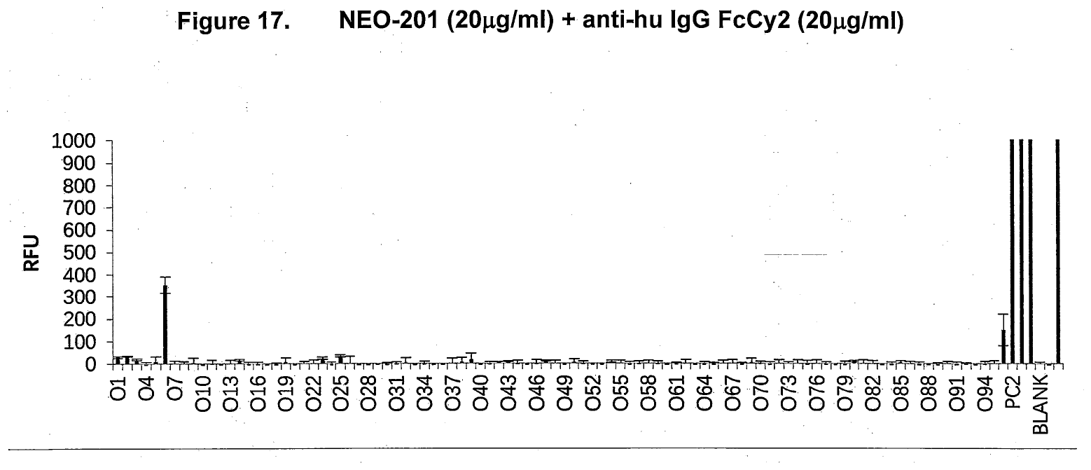

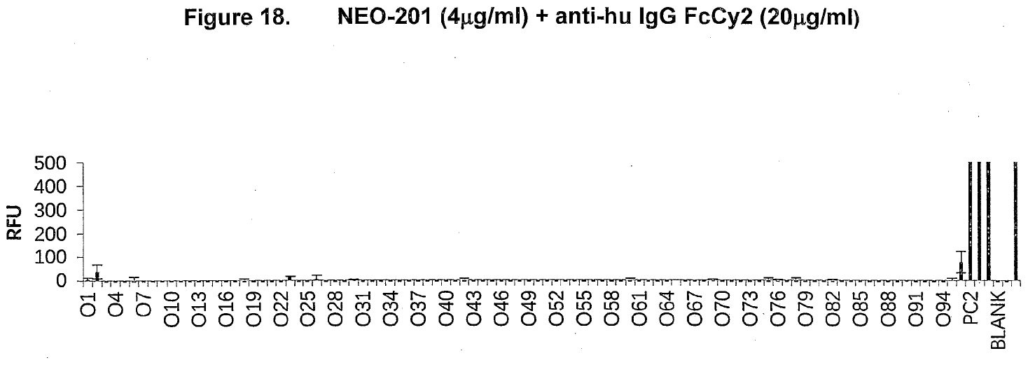

The background signal is low across the array. Binding patterns are clear with the anti-human lgG Fc Cy3 antibody detection method. This method shows that the binding events with O-glycans 01, 2, 6, 23, 26 and 39 seen with the anti-human lgG1 antibody method may be real binding events, and not non-specific binding (FIGS. 16, 17, 18, 19).

These results show that NEO-201 interacts with O-glycans 01, 02, 06, 023, 026 and 039 in the O-glycan Array. The 06 binding interaction was the strongest of any observed. 01 and 02 are Tn antigens. 06 is a Core 1, 023 is a Core 2, 026 is a Core 3 and 039 is a Core 4 O-glycan.

### O-Glycan Profiles of Tumor Cell Lines and Human Neutrophils

### Human Pancreatic Cancer Cell Line (CFPAC-1):

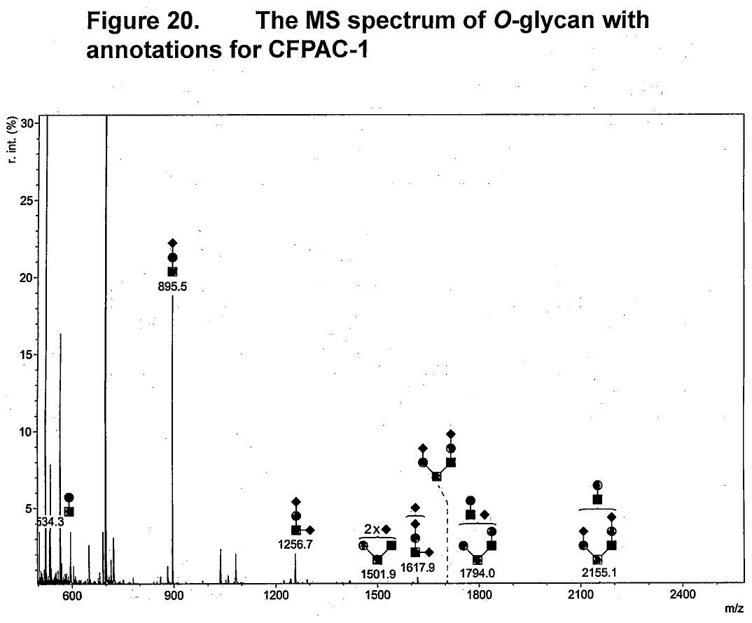

CFPAC-1 O-glycan profile shows a nice array of, mostly, sialylated O-glycans. Up to 10 O-glycans were identified for this sample. The following table summarizes the O-glycan profiled for CFPAC-1, including m/z, compositions, proposed structures, and relative abundance (FIG. 20 and FIG. 5). CFPAC-1 shows mostly Core 1 profile.

### Human Neutrophils:

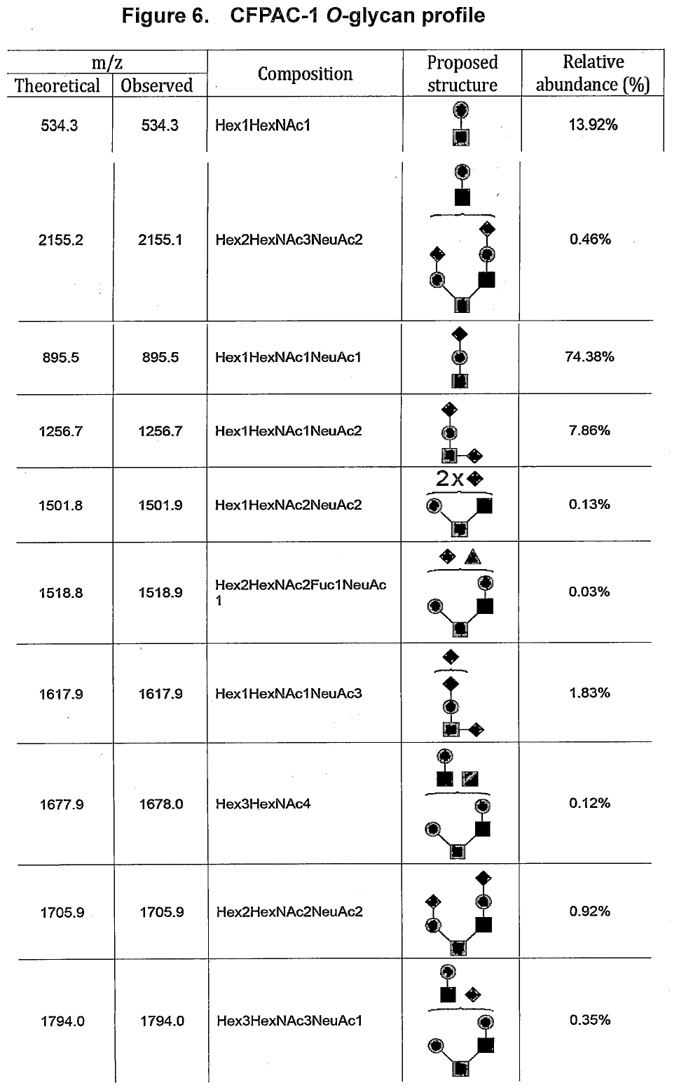

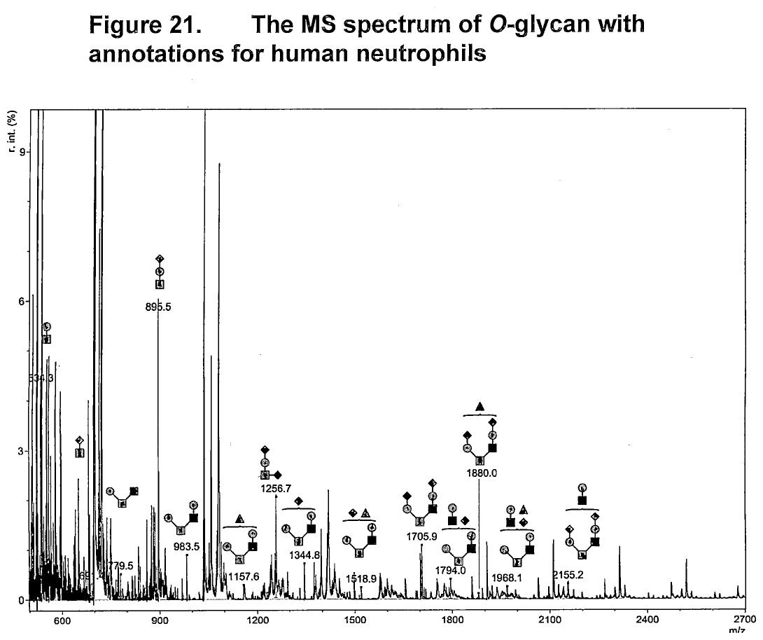

FIG. 21 and FIG. 6 show the O-glycan profile of human neutrophils. In comparison with CFPAC-1, there are significant numbers of peaks are common in two samples. But the relative abundancy varies.

### Human Hematological Neoplastic Cells: AML (HL60 and U937) and CML (K562)

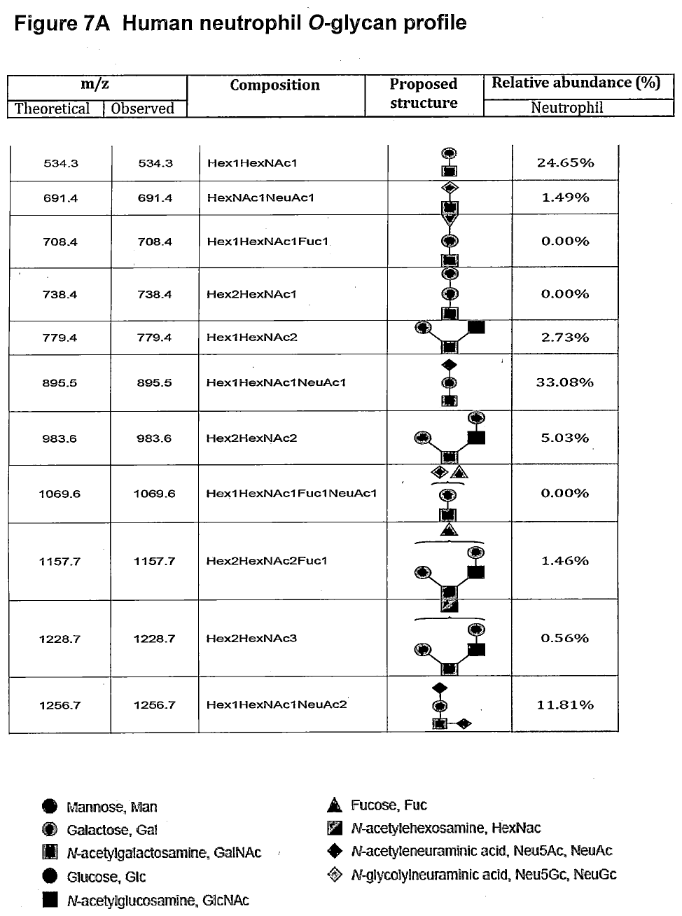

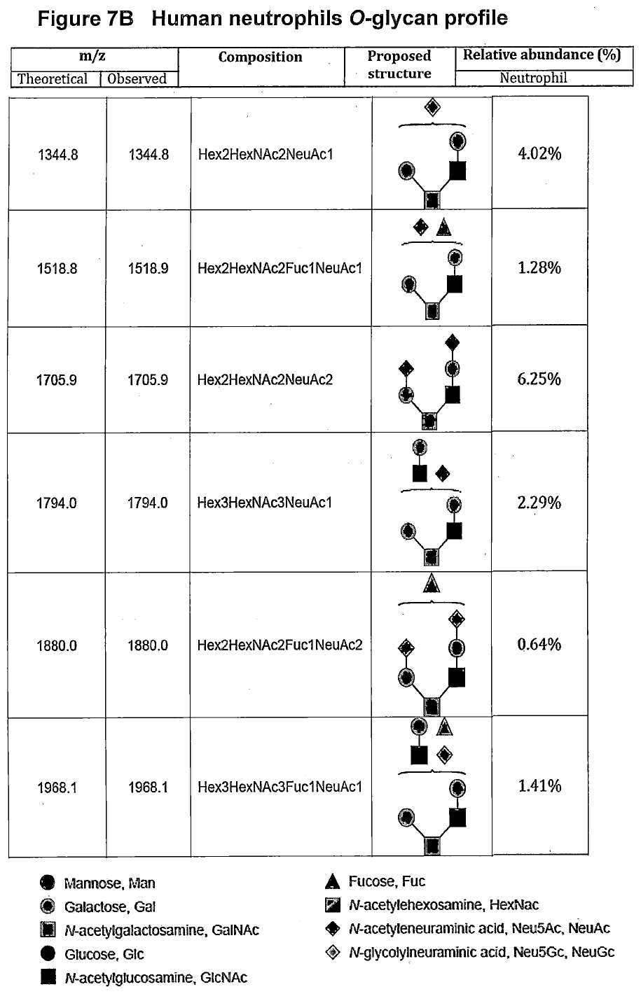

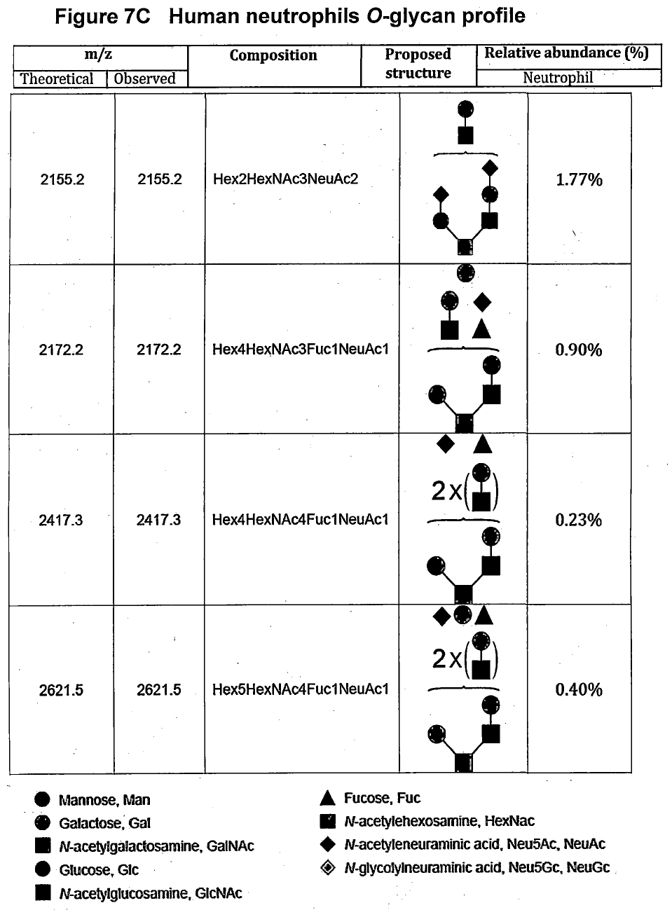

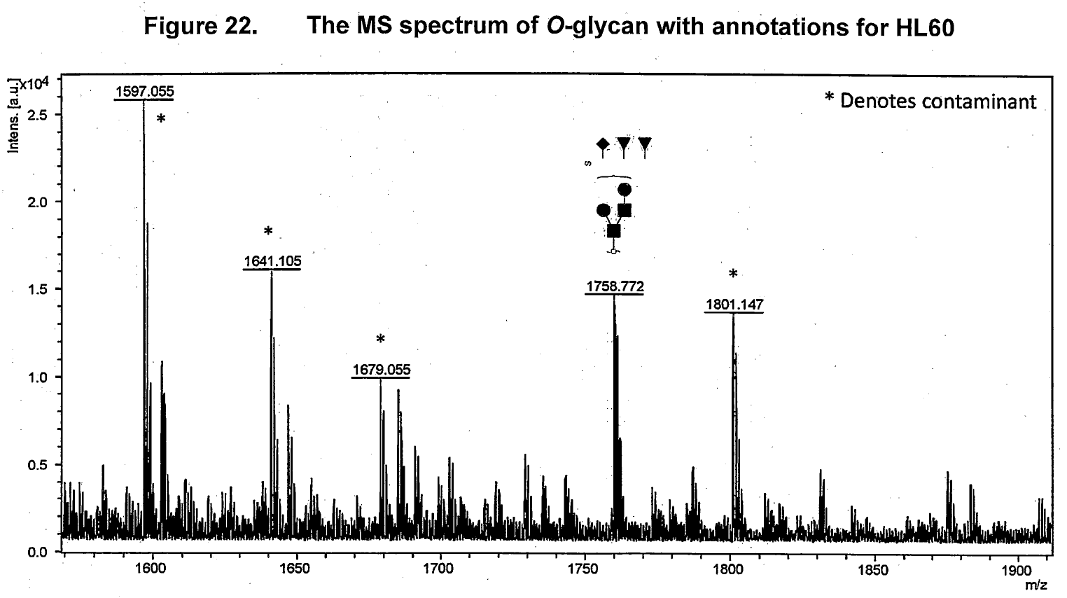

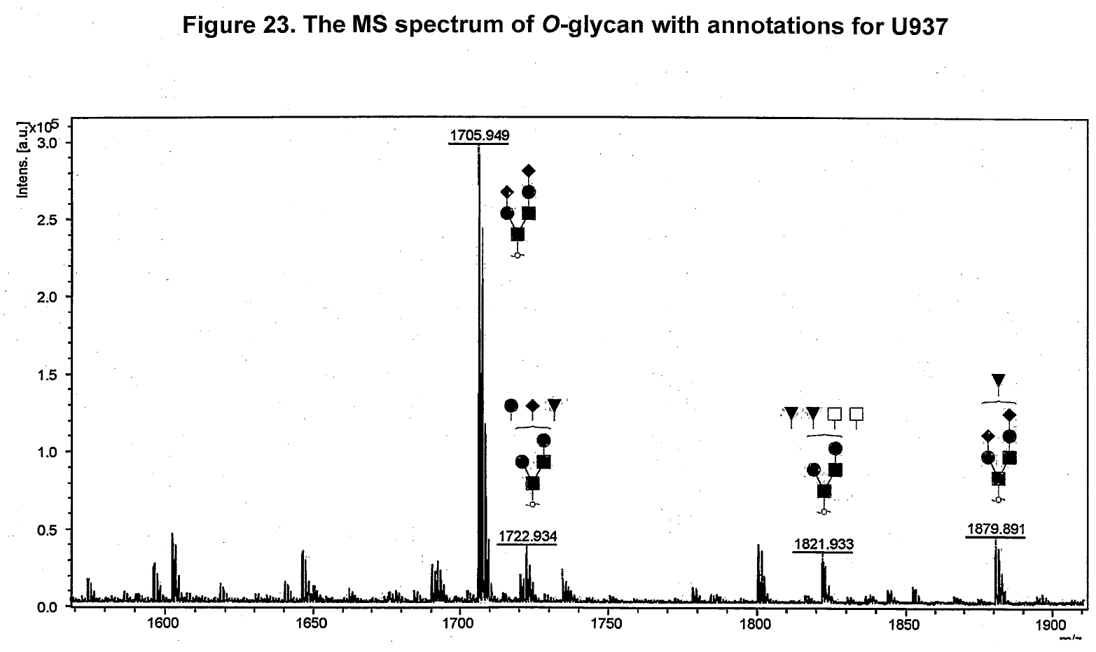

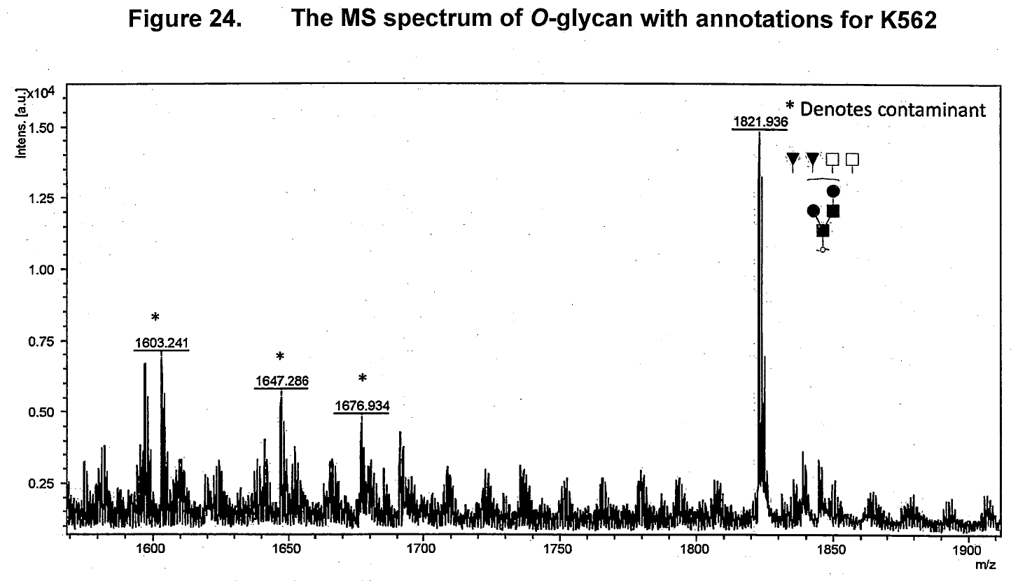

FIG. 22, FIG. 23, FIG. 24 and FIG. 7 show the O-glycan profile of the AML cell lines HL60 and U937 and the CML cell line K562 respectively. It can be seen that HL60 shows mostly an extended Core 1 profile, whereas U937 shows mostly extended Core 1 and Core 2 profiles and K562 shows mostly an extended Core 2 profile.

### NEO-201 Binding Affinity to C-Terminus Truncated CEACAM6 Protein in Mammalian Expression System

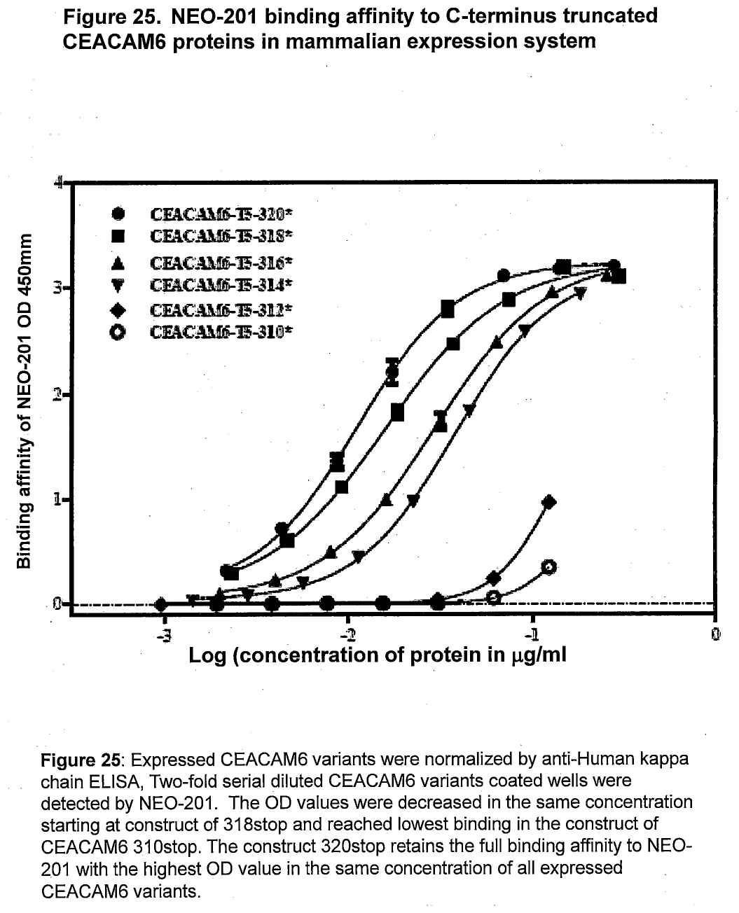

The supernatants after 48-72 hours transfection of the 293T cell line were collected and the secreted CEACAM6 in the supernatants was measured by ELISA using anti-human kappa chain antibody for quantification. The binding activity of NEO-201 to CEACAM6 truncated variants is presented in FIG. 25. It can be seen that the affinity of binding of NEO-201 gradually decreases with truncation of the C-terminus of CEACAM6. These results demonstrate that the shortest CEACAM6 sequence which retains complete binding affinity to NEO-201 ends at 319S of the CEACAM6 C-terminus (320Gstop construct). No binding was detected at 312T of the CEACAMC-terminus (312Tstop construct). The amino acid residues spanning 312 to 320 of CEACAM6 are TVTMITVSG. Since there are threonine residues at each of positions 312, 314 and 317 as well as a serine residue at position 319, GalNAc residues potentially can be added onto one or more of these threonine and serine residues to form O-glycans. This result is in accord with our finding that NEO-201 binds to an O-glycan comprising epitope of CEACAM6.

### NEO-201 Binding Affinity to C-Terminus Truncated CEACAM5 Protein in Mammalian Expression System

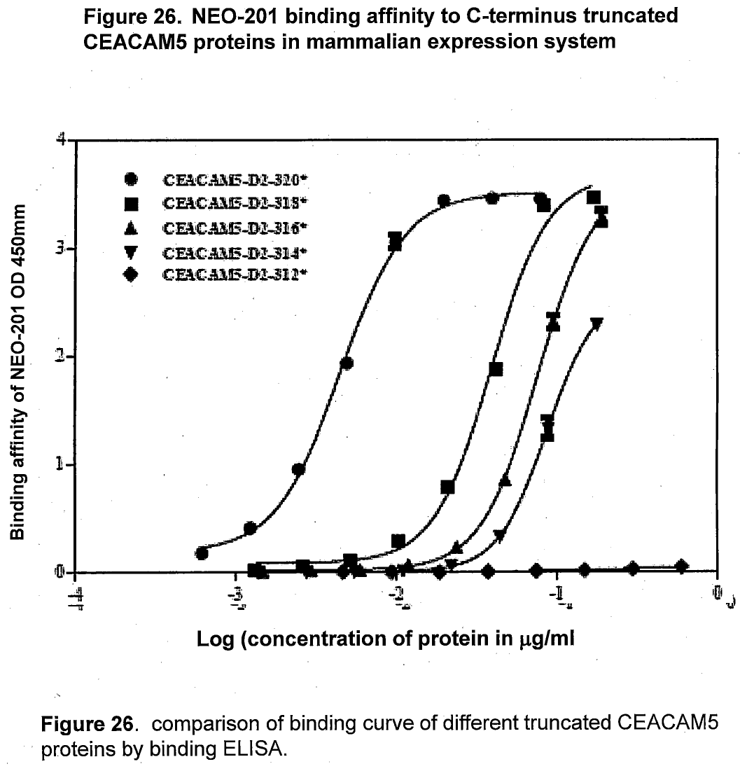

The binding curve from the truncated constructs CEACAM5-D2-320*, CEACAM5-D2-318*, CEACAM5-D2-316*, CEACAM5-D2-314* and CEACAM5-D2-312* shows that the CEACAM5-D2-320* protein retains the ability to specifically bind NEO-201. As can be seen from the results in FIG. 26 the binding activity gradually decreases with more truncation of amino acids in the C-terminus of CEACAM5 and there is no binding activity detected after deleting amino acid residues 311 to 685 (CEACAM5-D2-312*). These results demonstrate that the region of CEACAM5 spanning residues 310 to 318 is essential for the binding of NEO-201 to CEACAM5. The amino acids which comprise residues 310 to 318 of CEACAM5 inclusive are RTTVTTITV. As there is a threonine residue at each of positions 311, 312, 314, 315 and 317, GalNAc residues potentially can be added onto one or more of these threonine residues to form O-glycans. This result confirms our finding that NEO-201 binds to an O-glycan comprising epitope on CEACAM5.

### NEO-201 Reactivity Difference Between K562 (CML Cell Line) and CFPAC-1 (Pancreatic Cell Line) by Flow Cytometry

The O-glycan Array showed that NEO-201 interacts with 01, 02, 06, 023, 026 and 039 O-glycans and that 06 (Core 1) binding interaction was the strongest of any observed. To confirm that Core 1 glycans represent O-glycans with the strongest reactivity to NEO-201 we compared NEO-201 reactivity between CFPAC-1 (pancreatic cell line expressing high levels of Core 1 glycans, shown in FIG. 5) and K562 (Chronic Myeloid Leukemia cell line expressing only Core 2 glycans, shown in FIG. 8) by flow cytometry.

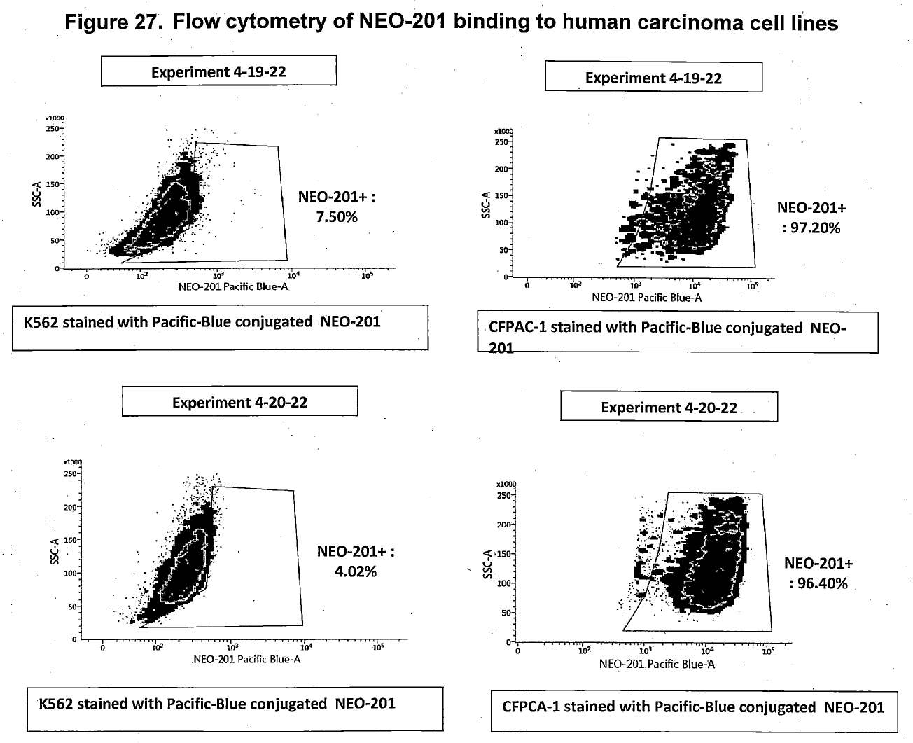

As shown in FIG. 27, NEO-201 did not react with K562 cells. Conversely, almost 100% of CFPAC-1 cells stained positively for NEO-201.

### CONCLUSIONS

The aberrant O-glycans expression at the cancer cell surface is an important post-translation modification of proteins and lipids and is strongly affected by oncogenesis. All these aberrant O-glycans may serve as potential targets to improve the diagnosis and treatment of tumors. Monoclonal antibodies that specifically recognize the Tn and T antigens have been widely used to detect malignant cells.

Our results show that NEO-201 interacts with O-glycans 01, 02, 06, 023, 026 and 039 O-glycans and that the strongest binding was observed with 06, 01 and 02 which are Tn antigens. 06 is Core 1, 023 is Core 2, 026 is Core 3 and 039 is Core 4.

NEO-201 reactive cells such as CFPAC-1, human neutrophils, human hematological neoplastic cells HL60, U937 shows expression of Core 1 and Core 2 O-glycan and/or the extended Core 1 and Core 2 O-glycan profiles.

To confirm the observation that Core 1 glycans show the strongest binding to NEO-201 we compared NEO-201 reactivity to CFPAC-1 cells (Core 1 glycans highly expressed on their surface) with K562 cells (express only Core 2 glycans) by flow cytometry.

As shown, we observed that NEO-201 did not react to K562 cells, expressing only extended Core 2 glycans. On the other hand, CFPAC-1, which expressed high levels of Core 1 (especially 06) and extended Core 1, showed high percentage of NEO-201 positive cells in flow cytometry. This observation suggests that NEO-201 binds strongly to Core 1 and/or extended Core 1 glycans. These results also confirm our finding that NEO-201 binds to mammalian expressed rhCEACAM6 but not bacterial expressed rhCEACMA6 (FIG. 6) because the mammalian expressed rhCEACAM6 expresses O-glycans not present on bacterial expressed rhCEACMA6.

We have identified the NEO-201 binding area of CEACAM6 and CEACAM5 from the truncation study. These results indicate that the amino acid sequence from 312 to 320 (TVTMITVSG) in CEACAM6 and the area of amino acid sequence from 310 to 318 (RTTVTTITV) in CEACAM5 are essential for the binding to NEO-201. These regions consist of threonine and serine residues and it is known that GalNAc residue can be added onto threonine to form O-glycans. These results together with the results from the O-glycan Array and O-glycan profiling from the NEO-201 reactive cells highly suggest that NEO-201 bind to the Core 1, Core 2 and/or the extended Core 1 and Core 2 in these areas of the protein sequence, but very likely Core 1 and/or the extended Core 1 are glycans with the highest binding to NEO-201.

### Humanized NEO-201 Monoclonal Antibody

The NEO-201 antibody sequences used in these examples are as shown below:

### H16C3-Abb* Heavy Chain:

(SEQ ID NO: 28)
MGWSCHFFLVATATGVHS/QVQLVQSGAEVKKPGASVKVSCKASGYTFT
DYAMHWVRQAPGQRLEWMGLISTYSGDTKYNQNFQGRVTMTVDKSASTA
YMELSSLRSEDTAVYYCARGDYSGSRYWFAYWGQGTLVTVSS/ASTKGP
SVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCD
KTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRW
QQGNVFSCSVMHEALHNHYTQKSLSLSPGK

### H16C3-Abb* Light Chain:

(SEQ ID NO: 29)
MGVPTQLLLLWLTVVVVRC/DIQMTQSPSSLSASVGDRVTITCQASENI
YGALNWYQRKPGKSPKLLIYGASNLATGMPSRFSGSGSGTDYTFTISSL
QPEDIATYYCQQVLSSPYTFGGGTKLEIKR/TVAAPSVFIFPPSDEQLK
SGTASVVCLLNNFYPREAKVQWKVDNALQSGNSQESVTEQDSKDSTYSL
SSTLTLSKADYEKHKVYACEVTHQGLSSPVTKSFNRGEC

The boundaries between the expression leader sequence, variable region, and constant region is delimited by a forward slash (“/”) in each sequence, and CDR sequences are shown in bold, underlined text. The antibody sequences used included the variable and constant regions shown. These include the heavy chain CDR1 of SEQ ID NO: 32, the heavy chain CDR2 of SEQ ID NO: 33, the heavy chain CDR3 of SEQ ID NO: 34, the light chain CDR1 of SEQ ID NO: 35, the light chain CDR2 of SEQ ID NO: 36, and the light chain CDR3 of SEQ ID NO: 37.

Each document cited herein is hereby incorporated by reference in its entirety.

1. A method of treating or preventing a hematological malignancy in a patient in need thereof, comprising administering to the patient an effective amount of an antibody or antibody fragment which binds to glycosylated CEACAM5 and CEACAM6 but not to aglycosylated CEACAM5 or aglycosylated CEACAM6.

2. The method of claim 1, wherein said antibody or antibody fragment recognizes an O-glycosylated epitope binding to the Threonine in the region of amino acids 310 to 318 (RTTVTTITV) of CEACAM5 and to the Threonine and Serine in the region of amino acids 312 to 320 (TVTMITVSG) of CEACAM6.

3. A method of treating or preventing a hematological malignancy in a patient in need thereof, comprising administering to the patient an effective amount of NEO-201 or an antigen binding fragment thereof.

4. The method of any one of the previous claims, wherein the hematological malignancy is characterized by cancer cells which express O-glycans selected from one or more of 01, 02, 06, 023, 026 and 039 O-glycans having the structure shown in the array in FIG. 11 and in FIG. 19.

5. The method of any one of the previous claims, wherein the hematological malignancy is characterized by cancer cells which express 06, 01 or 02 O-glycans having the structure shown in the array in FIG. 11 and structures in FIG. 19.

6. The method of any one of the previous claims, wherein the hematological malignancy is characterized by cancer cells which express 06 O-glycans as shown in the array in FIG. 11 and in FIG. 19.

7. The method any one of the previous claims, wherein the hematological malignancy is characterized by cancer cells which express Tn antigens or Core 1, 2, 3 or 4 O-glycans having the structures shown in FIG. 5.

8. The method of any one of the previous claims, wherein said hematological malignancy is selected from a leukemia, lymphoma, multiple myeloma, or myelodysplastic syndrome.

9. The method of any one of the previous claims, wherein said hematological malignancy is selected from acute lymphocytic leukemia (ALL), acute myeloid leukemia (AML), Burkitt lymphoma (BL), chronic lymphocytic leukemia (CLL), chronic myeloid leukemia (CML), hairy cell leukemia (HCL), Hodgkin's lymphoma (HL), marginal zone lymphoma (MZL), multiple myeloma (MM), myelodysplastic syndromes (MDS), myeloma, non-Hodgkin's lymphoma (NHL), or T-cell lymphoma (TCL).

10. The method of any one of the previous claims, wherein said hematological malignancy is a leukemia.

11. The method of claim 8, wherein said hematological malignancy is acute myeloid leukemia (AML), optionally acute promyelocytic leukemia (APL).

12. The method of any one of the previous claims, wherein said hematological malignancy is multiple myeloma.

13. The method of any one of the previous claims, wherein said hematological malignancy expresses CEACAM5 and/or CEACAM6.

14. The method of any one of the previous claims, further comprising, prior to or at the time of said administering said antibody:
(i) determining that said hematological malignancy is CEACAM5 and/or CEACAM6 positive;
(ii) determining that said hematological malignancy expresses (i) O-glycans selected from one or more of 01, 02, 06, 023, 026 and 039 having the structure shown in the array in FIG. 11 and in FIG. 19,
(iii) 06, 01 or 02 O-glycans having the structure shown in the array in FIG. 11 and in FIG. 19,
(iv) 06 O-glycans having the structure shown in the array in FIG. 11 and in FIG. 19 and/or
(v) Tn antigens or Core 1, 2, 3 or 4 O-glycans having the structures shown in FIG. 5.

15. The method of any one of the foregoing claims, wherein said NEO-201 antibody:
(i) comprises the VH and VL CDR sequences contained in SEQ ID NO: 28 and SEQ ID NO: 29.
(ii) comprises a variable heavy chain sequence having at least 90% identity to SEQ ID NO: 38;
(iii) comprises a variable light chain sequence having at least 90% identity to SEQ ID NO: 39;
(iv) comprises a variable heavy chain sequence having at least 90% identity to SEQ ID NO: 38 and a variable light chain sequence having at least 90% identity to SEQ ID NO: 39;
(v) comprises a variable heavy chain sequence having at least 95% identity to SEQ ID NO: 38 and a variable light chain sequence having at least 95% identity to SEQ ID NO: 39;
(vi) comprises a variable heavy chain sequence identical to SEQ ID NO: 38 and a variable light chain sequence identical to SEQ ID NO: 39;
(vii) comprises a heavy chain sequence having at least 90% identity to amino acids 20-470 of SEQ ID NO: 28 and a light chain sequence having at least 90% identity to amino acids 20-233 of SEQ ID NO: 29;
(viii) comprises a heavy chain sequence having at least 95% identity to amino acids 20-470 of SEQ ID NO: 28 and a light chain sequence having at least 95% identity to amino acids 20-233 of SEQ ID NO: 29;
(ix) comprises a heavy chain sequence identical to amino acids 20-470 of SEQ ID NO: 28 and a light chain sequence identical to amino acids 20-233 of SEQ ID NO: 29;
(x) consists of the heavy chain sequence of amino acids 20-470 of SEQ ID NO: 28 and the light chain sequence of amino acids 20-233 of SEQ ID NO: 29;
(xi) comprises a human lgG1 constant domain;
(xii) is humanized;
(xiii) is conjugated to another moiety;
(xiv) is conjugated to another cytotoxic moiety, label, radioactive moiety, or affinity tag; or
(xv) is comprised in a chimeric antigen receptor (CAR);
(xvi) said antibody is a multispecific or bispecific antibody which targets at least one other antigen, optionally another tumor antigen or an antigen expressed on an immune cell, optionally wherein said other antigen is a checkpoint inhibitor or cytokine or hormone or growth factor.

16. The method of any one of the foregoing claims, wherein said antibody is administered as an immune cell, optionally a T or NK cell, which immune cell expresses a CAR comprising said antibody.

17. A method of killing hematological malignancy cells in vivo, comprising administering an effective amount of a NEO-201 antibody to a patient.

18. A method of killing hematological malignancy cells in vivo, comprising administering an effective amount of a NEO-201 antibody to a patient and at least one other active agent, wherein NEO-201 and the and at least one other active agent elicit an additive or synergistic effect in killing hematological malignancy cells.

19. A method of treating or preventing a hematological malignancy, decreasing the burden of a hematological malignancy, or slowing the growth or proliferation rate of a hematological malignancy, comprising administering an effective amount of a NEO-201 antibody to a patient in need thereof.

20. The method of any one of claims 17-19, wherein said hematological malignancy is selected from leukemia, lymphoma, multiple myeloma, or myelodysplastic syndromes.

21. The method of claim 20, wherein said hematological malignancy is selected from acute lymphocytic leukemia (ALL), acute myeloid leukemia (AML), Burkitt lymphoma (BL), chronic lymphocytic leukemia (CLL), chronic myeloid leukemia (CML), hairy cell leukemia (HCL), Hodgkin's lymphoma (HL), marginal zone lymphoma (MZL), multiple myeloma (MM), myelodysplastic syndromes (MDS), myeloma, non-Hodgkin's lymphoma (NHL), or T-cell lymphoma (TCL).

22. The method of claim 20, wherein said hematological malignancy is AML and includes M0 (Undifferentiated acute myeloblastic leukemia), M1 (Acute myeloblastic leukemia with minimal maturation), M2 (Acute myeloblastic leukemia with maturation), M3 (Acute promyelocytic leukemia (APL)), M4 (Acute myelomonocytic leukemia), M4 eos (Acute myelomonocytic leukemia with eosinophilia), M5 (Acute monocytic leukemia), M6 (Acute erythroid leukemia) and M7 (Acute megakaryoblastic leukemia), AML with certain genetic abnormalities (gene or chromosome changes), AML with a translocation between chromosomes 8 and 21, AML with a translocation or inversion in chromosome 16, APL with the PML-RARA fusion gene, AML with a translocation between chromosomes 9 and 11, AML with a translocation between chromosomes 6 and 9, AML with a translocation or inversion in chromosome 3, AML (megakaryoblastic) with a translocation between chromosomes 1 and 22, AML with the BCR-ABL1 (BCR-ABL) fusion gene, AML with mutated NPM1 gene, AML with biallelic mutations of the CEBPA gene (i.e., mutations in both copies of the gene), AML with mutated RUNX1 gene, AML with myelodysplasia-related changes, AML related to previous chemotherapy or radiation, AML with minimal differentiation (FAB M0), AML without maturation (FAB M1), AML with maturation (FAB M2), Acute myelomonocytic leukemia (FAB M4), Acute monoblastic/monocytic leukemia (FAB M5), Pure erythroid leukemia (FAB M6), Acute megakaryoblastic leukemia (FAB M7), Acute basophilic leukemia, Acute panmyelosis with fibrosis, Myeloid sarcoma (also known as granulocytic sarcoma or chloroma), and Myeloid proliferations related to Down syndrome.

23. The method of claim 20, wherein said hematological malignancy is an AML-like condition e.g., an undifferentiated or bi-phenotypic acute leukemia or a mixed phenotype acute leukemia (MPAL).

24. The method of claim 20, wherein said hematological malignancy is acute myeloid leukemia (AML), further optionally acute promyelocytic leukemia.

25. The method of claim 20, wherein said hematological malignancy is myeloma.

26. The method of any one of claims 17-25, wherein said hematological malignancy:
(i) expresses CEACAM5 or CEACAM6;
(ii) expresses O-glycans selected from one or more of 01, 02, 06, 023, 026 and 039 O-glycans having the structure shown in the array in FIG. 11 and in FIG. 19;
(iii) expresses 06, 01 or 02 O-glycans having the structure shown in the array in FIG. 11 and structures in FIG. 19;
(iv) expresses 06 O-glycans as shown in the array in FIG. 11 and in FIG. 19 and/or
(v) expresses Tn antigens or Core 1, 2, 3 or 4 O-glycans having the structures shown in FIG. 5.

27. The method of any one of claims 17-26, wherein, further comprising, prior to or at the time of said administering, determining that said hematological malignancy:
(i) expresses CEACAM5 or CEACAM6;
(ii) expresses O-glycans selected from one or more of 01, 02, 06, 023, 026 and 039 O-glycans having the structure shown in the array in FIG. 11 and in FIG. 19;
(iii) expresses 06, 01 or 02 O-glycans having the structure shown in the array in FIG. 11 and structures in FIG. 19; or
(iv) expresses 06 O-glycans as shown in the array in FIG. 11 and in FIG. 19 and/or
(v) expresses Tn antigens or Core 1, 2, 3 or 4 O-glycans having the structures shown in FIG. 5.

28. The method of any one of claims 17-27, wherein:
(i) comprises the VH and VL CDR sequences contained in SEQ ID NO: 28 and SEQ ID NO: 29;
(ii) comprises a variable heavy chain sequence having at least 90% identity to SEQ ID NO: 38;
(iii) comprises a variable light chain sequence having at least 90% identity to SEQ ID NO: 39;
(iv) comprises a variable heavy chain sequence having at least 90% identity to SEQ ID NO: 38 and a variable light chain sequence having at least 90% identity to SEQ ID NO: 39;
(v) comprises a variable heavy chain sequence having at least 95% identity to SEQ ID NO: 38 and a variable light chain sequence having at least 95% identity to SEQ ID NO: 39;
(vi) comprises a variable heavy chain sequence identical to SEQ ID NO: 38 and a variable light chain sequence identical to SEQ ID NO: 39;
(vii) comprises a heavy chain sequence having at least 90% identity to amino acids 20-470 of SEQ ID NO: 28 and a light chain sequence having at least 90% identity to amino acids 20-233 of SEQ ID NO: 29;
(viii) comprises a heavy chain sequence having at least 95% identity to amino acids 20-470 of SEQ ID NO: 28 and a light chain sequence having at least 95% identity to amino acids 20-233 of SEQ ID NO: 29;
(ix) comprises a heavy chain sequence identical to amino acids 20-470 of SEQ ID NO: 28 and a light chain sequence identical to amino acids 20-233 of SEQ ID NO: 29;
(x) consists of the heavy chain sequence of amino acids 20-470 of SEQ ID NO: 28 and the light chain sequence of amino acids 20-233 of SEQ ID NO: 29;
(xi) comprises a human IgG1 constant domain;
(xii) is humanized;
(xiii) is conjugated to another moiety;
(xiv) is conjugated to another cytotoxic moiety, label, radioactive moiety, or affinity tag;
(xv) is comprised in a chimeric antigen receptor (CAR); or
(xvi) said antibody is a multispecific or bispecific antibody which targets at least one other antigen, optionally another tumor antigen or an antigen expressed on an immune cell, optionally wherein said other antigen is a checkpoint inhibitor or cytokine or hormone or growth factor.

29. The method of any one of the foregoing claims, further comprising administering another therapeutic agent to said patient.

30. The method of claim 29, wherein said other agent is selected from (a) microtubule inhibitors, topoisomerase inhibitors, platins, alkylating agents, and anti-metabolites; (b) MK-2206, ON 013105, RTA 402, BI 2536, Sorafenib, ISIS-STAT3Rx, a microtubule inhibitor, a topoisomerase inhibitor, a platin, an alkylating agent, an anti-metabolite, paclitaxel, gemcitabine, doxorubicin, vinblastine, etoposide, 5-fluorouracil, carboplatin, altretamine, aminoglutethimide, amsacrine, anastrozole, azacitidine, bleomycin, busulfan, carmustine, chlorambucil, 2-chlorodeoxyadenosine, cisplatin, colchicine, cyclophosphamide, cytarabine, cytoxan, dacarbazine, dactinomycin, daunorubicin, docetaxel, estramustine phosphate, floxuridine, fludarabine, gentuzumab, hexamethylmelamine, hydroxyurea, ifosfamide, imatinib, interferon, irinotecan, lomustine, mechlorethamine, melphalen, 6-mercaptopurine, methotrexate, mitomycin, mitotane, mitoxantrone, pentostatin, procarbazine, rituximab, streptozocin, tamoxifen, temozolomide, teniposide, 6-thioguanine, topotecan, trastuzumab, vincristine, vindesine, and/or vinorelbine; (c) 1-D-ribofuranosyl-1,2,4-triazole-3 carboxamide, 9->2-hydroxy-ethoxy methylguanine, adamantanamine, 5-iodo-2′-deoxyuridine, trifluorothymidine, interferon, adenine arabinoside, protease inhibitors, thymidine kinase inhibitors, sugar or glycoprotein synthesis inhibitors, structural protein synthesis inhibitors, attachment and adsorption inhibitors, and nucleoside analogues such as acyclovir, penciclovir, valacyclovir, and ganciclovir; (d) a PD-1 inhibitor or anti-PD-1 antibody such as KEYTRUDA® (pembrolizumab), OPDIVO® (nivolumab), or LIBTAYO (cemiplimab); (e) a PD-L1 inhibitor or anti-PD-L1 antibody such as TECENTRIQ (atezolizumab), IMFINZI (durvalumab), or BAVENCIO (avelumab); or (f) a CTLA-4 inhibitor or anti-CTLA-4 antibody such as YERVOY® ipilimumab.

31. The method of any one of the foregoing claims, further comprising administering an anti-cancer vaccine to said patient.

32. The method of any one of the foregoing claims, wherein said hematological malignancy cells are killed by CDC and/or ADCC.

33. The method of any one of the foregoing claims, wherein said NEO-201 antibody is coupled to a cytotoxic moiety.

34. A method of killing hematological malignancy cells in vitro, comprising contacting said hematological malignancy cells with a NEO-201 antibody.

35. The method of claim 34, further comprising one or more of the following:
(i) contacting said hematological malignancy cells with complement; or
(ii) contacting said hematological malignancy cells with effector cells, optionally natural killer cells.

36. A method of detecting hematological malignancy cells, comprising detecting the expression of the NEO-201 antigen by said hematological malignancy cells, optionally wherein the level of hematological malignancy cells in a patient sample, such as a blood or biopsy sample, is used to diagnose cancer or determine cancer prognosis, wherein optionally (i) said NEO-201 antibody is directly or indirectly coupled to a label, (ii) said detecting comprises cell sorting, further optionally fluorescence activated cell sorting.

37. A method of staining hematological malignancy cells, comprising contacting cells with a NEO-201 antibody, wherein optionally said NEO-201 antibody is directly or indirectly coupled to a label.

38. A method of isolating hematological malignancy cells, comprising isolating cells that express the NEO-201 antigen, which optionally comprises contacting a sample containing hematological malignancy cells with a NEO-201 antibody, further optionally wherein said NEO-201 antibody is directly or indirectly labeled.

39. The method of claim 38, wherein said sample is or comprises blood or bone marrow or tumor biopsy sample.

40. The method of claim 38 or 39, comprising separating NEO-201 positive hematological malignancy cells from NEO-201 negative cells, optionally by cell sorting, further optionally fluorescence activated cell sorting.

41. The method of claim 38 or 39, wherein said hematological malignancy cells are isolated by contacting sample with a support comprising a NEO-201 antibody, whereby said hematological malignancy cells are retained on said support.

42. The method of any one of the prior claims, wherein said NEO-201 antibody:
(i) comprises the VH and VL CDR sequences contained in SEQ ID NO: 28 and SEQ ID NO: 29;
(ii) comprises a variable heavy chain sequence having at least 90% identity to SEQ ID NO: 38;
(iii) comprises a variable light chain sequence having at least 90% identity to SEQ ID NO: 39;
(iv) comprises a variable heavy chain sequence having at least 90% identity to SEQ ID NO: 38 and a variable light chain sequence having at least 90% identity to SEQ ID NO: 39;
(v) comprises a variable heavy chain sequence having at least 95% identity to SEQ ID NO: 38 and a variable light chain sequence having at least 95% identity to SEQ ID NO: 39;
(vi) comprises a variable heavy chain sequence identical to SEQ ID NO: 38 and a variable light chain sequence identical to SEQ ID NO: 39;
(vii) comprises a heavy chain sequence having at least 90% identity to amino acids 20-470 of SEQ ID NO: 28 and a light chain sequence having at least 90% identity to amino acids 20-233 of SEQ ID NO: 29;
(viii) comprises a heavy chain sequence having at least 95% identity to amino acids 20-470 of SEQ ID NO: 28 and a light chain sequence having at least 95% identity to amino acids 20-233 of SEQ ID NO: 29;
(ix) comprises a heavy chain sequence identical to amino acids 20-470 of SEQ ID NO: 28 and a light chain sequence identical to amino acids 20-233 of SEQ ID NO: 29;
(x) consists of the heavy chain sequence of amino acids 20-470 of SEQ ID NO: 28 and the light chain sequence of amino acids 20-233 of SEQ ID NO: 29;
(xi) comprises a human lgG1 constant domain;
(xii) is humanized;
(xiii) is conjugated to another moiety;
(xiv) is conjugated to another cytotoxic moiety, label, radioactive moiety, or affinity tag; (xv) is comprised in a chimeric antigen receptor (CAR); or
(xvi) said antibody is a multispecific or bispecific antibody which targets at least one other antigen, optionally another tumor antigen or an antigen expressed on an immune cell, optionally wherein said other antigen is a checkpoint inhibitor or cytokine or hormone or growth factor.


SEQUENCE LISTING (39 sequences)

SEQ ID NO: 1 - protein, 11 aa, Mus musculus
GASENIYGALN

SEQ ID NO: 2 - protein, 7 aa, Mus musculus
GASNLAD

SEQ ID NO: 3 - protein, 9 aa, Mus musculus
QNVLSSPYT

SEQ ID NO: 4 - protein, 11 aa, Homo sapiens
QASENIYGALN

SEQ ID NO: 5 - protein, 7 aa, Homo sapiens
GASNLAT

SEQ ID NO: 6 - protein, 9 aa, Homo sapiens
QQVLSSPYT

SEQ ID NO: 7 - protein, 10 aa, Mus musculus
GYTFTDYAMH

SEQ ID NO: 8 - protein, 17 aa, Mus musculus
LISTYSGDTKYNQNFKG

SEQ ID NO: 9 - protein, 12 aa, Mus musculus
GDYSGSRYWFAY

SEQ ID NO: 10 - protein, 17 aa, Homo sapiens
LISTYSGDTKYNQNFQG

SEQ ID NO: 11 - protein, 12 aa, Homo sapiens
GDYSGSRYWFAY

SEQ ID NO: 12 - DNA, 965 bp, Mus musculus
gcggggcagcctcacacagaacacacacagatatgggtgtacccactcagctcctgttgctgtggcttacagtcgtagttgtcagatgtgacatccagatgactcagtctccagcttcactgtctgcatctgtgggagaaactgtcaccatcacatgtggagcaagtgagaatatttacggtgctttaaattggtatcagcggaaacagggaaaatctcctcagctcctgatttatggcgcaagtaatttggcagatggcatgtcatcgaggttcagtggcagtggatctggtagacagtattctctcaagatcagtagcctgcatcctgacgatgttgcaacgtattactgtcaaaatgtattaagtagtccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacaccaacttcacccattgtcaagagcttcaacaggaatgagtgttagagacaaaggtcctgagacgccaccaccagctccccagctccatcctatcttcccttctaaggtcttggaggcttccccacaagcgacctaccactgttgcggtgctccaaacctcctccccacctccttctcctcctcctccctttccttggcttttatcatgctaatatttgcagaaaatattcaataaagtgagtctttgcacaaaaaaaaaaaaaaaaaaaaaaaaaa

SEQ ID NO: 13 - DNA, 1575 bp, Mus musculus
acgcgggacacagtagtctctacagtcacaggagtacacaggacattgccatgggttggagctgtatcatcttctttctggtagcaacagctacaggtgtgcactcccaggtccagctgcagcagtctgggcctgaggtggtgaggcctggggtctcagtgaagatttcctgcaagggttccggctacacattcactgattatgctatgcactgggtgaagcagagtcatgcaaagagtctcgagtggattggacttattagtacttacagtggtgatacaaagtacaaccagaactttaagggcaaggccacaatgactgtagacaaatcctccaacacagcctatatggaacttgccagattgacatctgaggattctgccatctattactgtgcaagaggggattattccggtagtaggtactggtttgcttactggggccaagggactctggtcactgtctctgcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatgatcccagtgtccttggagccctctggccctacaggactttgacacctacctccacccctccctgtataaataaagcacccagcactgcctcgggaccctgcataaaaaaaaaaaaaaaaaaaaaaaaaaaa

SEQ ID NO: 14 - protein, 107 aa, Mus musculus
LMTQSPASLSASVGETVTITCGASENIYGALNWYQRKQGKSPQLLIYGASNLADGMSSRFSGSGSGRQYSLKISSLHPDDVATYYCQNVLSSPYTFGGGTKLEIKKG

SEQ ID NO: 15 - protein, 116 aa, Mus musculus
LEESGPEVVRPGVSVKISCKGSGYTFTDYAMHWVKQSHAKSLEWIGLISTYSGDTKYNQNFKGKATMTVDKSSNTAYMELARLTSEDSAIYYCARGDYSGSRYWFAYWGQGTTVTR

SEQ ID NO: 16 - protein, 107 aa, Mus musculus
DIQMTQSPASLSASVGETVTITCGASENIYGALNWYQRKQGKSPQLLIYGASNLADGMSSRFSGSGSGRQYSLKISSLHPDDVATYYCQNVLSSPYTFGGGTKLEIK

SEQ ID NO: 17 - protein, 107 aa, Artificial Sequence
  Humanized antibody sequence
DIQMTQSPSSLSASVGDRVTITCGASENIYGALNWYQRKPGKSPKLLIYGASNLADGMPSRFSGSGSGRQYTLTISSLQPEDVATYYCQNVLSSPYTFGGGTKLEIK

SEQ ID NO: 18 - protein, 107 aa, Artificial Sequence; identical to SEQ ID NO: 17
  Humanized antibody sequence

SEQ ID NO: 19 - protein, 107 aa, Artificial sequence
  Humanized antibody sequence
DIQMTQSPSSLSASVGDRVTITCQASENIYGALNWYQRKPGKSPKLLIYGASNLATGMPSRFSGSGSGTDYTFTISSLQPEDIATYYCQNVLSSPYTFGGGTKLEIK

SEQ ID NO: 20 - protein, 107 aa, Artificial sequence
  Humanized antibody sequence
DIQMTQSPSSLSASVGDRVTITCQASENIYGALNWYQRKPGKSPKLLIYGASNLATGMPSRFSGSGSGRQYTFTISSLQPEDIATYYCQQVLSSPYTFGGGTKLEIK

SEQ ID NO: 21 - protein, 107 aa, Artificial Sequence
  Humanized antibody sequence
DIQMTQSPSSLSASVGDRVTITCGASENIYGALNWYQRKPGKSPNLLIYGASNLADGMPSRFSGSGSGRQYTLTISSLQPEDVATYYCQNVLSSPYTFGGGTKLEIK

SEQ ID NO: 22 - protein, 121 aa, Mus musculus
QVQLQQSGPEVVRPGVSVKISCKGSGYTFTDYAMHWVKQSHAKSLEWIGLISTYSGDTKYNQNFKGKATMTVDKSSNTAYMELARLTSEDSAIYYCARGDYSGSRYWFAYWGQGTLVTVSA

SEQ ID NO: 23 - protein, 121 aa, Artificial sequence
  Humanized antibody sequence
QVQLVQSGAEVKKPGASVKVSCKGSGYTFTDYAMHWVRQAPGQRLEWIGLISTYSGDTKYNQNFKGKATMTVDKSASTAYMELSSLRSEDTAVYYCARGDYSGSRYWFAYWGQGTLVTVSS

SEQ ID NO: 24 - protein, 121 aa, Artificial sequence
  Humanized antibody sequence
QVQLVQSGAEVKKPGASVKVSCKGSGYTFTDYAMHWVRQAPGQGLEWIGLISTYSGDTKYNQNFKGKATMTVDTSISTAYMELSRLRSDDTAIYYCARGDYSGSRYWFAYWGQGTLVTVSS

SEQ ID NO: 25 - protein, 121 aa, Artificial sequence
  Humanized antibody sequence
QVQLVQSGAEVKKPGASVKVSCKASGYTFTDYAMHWVRQAPGQRLEWMGLISTYSGDTKYSQKFQGRVTMTVDKSASTAYMELSSLRSEDTAVYYCARGDYSGSRYWFAYWGQGTLVTVSS

SEQ ID NO: 26 - protein, 121 aa, Artificial sequence
  Humanized antibody sequence
QVQLVQSGAEVKKPGASVKVSCKASGYTFTDYAMHWVRQAPGQRLEWIGLISTYSGDTKYNQKFQGKATMTVDKSASTAYMELSSLRSEDTAVYYCARGDYSGSRYWFAYWGQGTLVTVSS

SEQ ID NO: 27 - protein, 121 aa, Artificial sequence
  Humanized antibody sequence
QVQLVQSGAEVKKPGASVKVSCKGSGYTFTDYAMHWVRQVHAQGLEWIGLISTYSGDTKYNQNFKGKATMTVDKSTSTAYMELSSLRSEDTAVYYCARGDYSGSRYWFAYWGQGTLVTVSS

SEQ ID NO: 28 - protein, 470 aa, Artificial sequence
  Humanized antibody sequence (h16C3-Abb* heavy chain)
  MISC_FEATURE (1)..(19): Leader sequence
  MISC_FEATURE (20)..(140): Variable region
  MISC_FEATURE (141)..(470): Constant region
  MISC_FEATURE (45)..(54): CDR1
  MISC_FEATURE (70)..(85): CDR2
  MISC_FEATURE (118)..(129): CDR3
MGWSCIIFFLVATATGVHSQVQLVQSGAEVKKPGASVKVSCKASGYTFTDYAMHWVRQAPGQRLEWMGLISTYSGDTKYNQNFQGRVTMTVDKSASTAYMELSSLRSEDTAVYYCARGDYSGSRYWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 233 aa, Artificial sequence
  Humanized antibody sequence (h16C3-Abb* light chain)
  MISC_FEATURE (1)..(19): Leader sequence
  MISC_FEATURE (20)..(127): Variable region
  MISC_FEATURE (128)..(233): Constant region
  MISC_FEATURE (43)..(53): CDR1
  MISC_FEATURE (69)..(75): CDR2
  MISC_FEATURE (108)..(116): CDR3
MGVPTQLLLLWLTVVVVRCDIQMTQSPSSLSASVGDRVTITCQASENIYGALNWYQRKPGKSPKLLIYGASNLATGMPSRFSGSGSGTDYTFTISSLQPEDIATYYCQQVLSSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 17 aa, Homo sapiens
LISTYSGDTKYSQKFQG

SEQ ID NO: 31 - protein, 17 aa, Artificial sequence
  Humanized antibody sequence
LISTYSGDTKYNQKFQG

SEQ ID NO: 32 - protein, 10 aa, Artificial sequence
  Humanized antibody sequence (h16C3-Abb* heavy chain CDR1)
GYTFTDYAMH

SEQ ID NO: 33 - protein, 16 aa, Artificial sequence
  Humanized antibody sequence (h16C3-Abb* heavy chain CDR2)
ISTYSGDTKYNQNFQG

SEQ ID NO: 34 - protein, 12 aa, Artificial sequence
  Humanized antibody sequence (h16C3-Abb* heavy chain CDR3)
GDYSGSRYWFAY

SEQ ID NO: 35 - protein, 11 aa, Artificial sequence
  Humanized antibody sequence (h16C3-Abb* light chain CDR1)
QASENIYGALN

SEQ ID NO: 36 - protein, 7 aa, Artificial sequence
  Humanized antibody sequence (h16C3-Abb* light chain CDR2)
GASNLAT

SEQ ID NO: 37 - protein, 9 aa, Artificial sequence
  Humanized antibody sequence (h16C3-Abb* light chain CDR3)
QQVLSSPYT

SEQ ID NO: 38 - protein, 121 aa, Artificial sequence
  Humanized antibody sequence (h16C3-Abb* heavy chain variable)
  MISC_FEATURE (26)..(35): CDR1
  MISC_FEATURE (51)..(66): CDR2
  MISC_FEATURE (99)..(110): CDR3
QVQLVQSGAEVKKPGASVKVSCKASGYTFTDYAMHWVRQAPGQRLEWMGLISTYSGDTKYNQNFQGRVTMTVDKSASTAYMELSSLRSEDTAVYYCARGDYSGSRYWFAYWGQGTLVTVSS

SEQ ID NO: 39 - protein, 108 aa, Artificial sequence
  Humanized antibody sequence (h16C3-Abb* light chain)
  MISC_FEATURE (24)..(34): CDR1
  MISC_FEATURE (50)..(56): CDR2
  MISC_FEATURE (89)..(97): CDR3
DIQMTQSPSSLSASVGDRVTITCQASENIYGALNWYQRKPGKSPKLLIYGASNLATGMPSRFSGSGSGTDYTFTISSLQPEDIATYYCQQVLSSPYTFGGGTKLEIKR